(19) United States
(12) Patent Application Publication
(10) Pub. No.: US 2019/0271703 A1
(43) Pub. Date: Sep. 5, 2019

(54) BIOMARKERS AND TREATMENTS FOR METASTATIC CANCER

(71) Applicant: DUKE UNIVERSITY, Durham, NC (US)

(72) Inventors: Robin E. Bachelder, Durham, NC (US); Gabi Hanna, Durham, NC (US); Greg Palmer, Durham, NC (US); Andrew J. Armstrong, Durham, NC (US); Donald P. McDonnell, Durham, NC (US)

(21) Appl. No.: 16/345,422

(22) PCT Filed: Oct. 26, 2017
(86) PCT No.: PCT/US17/58575

§ 371 Date: Apr. 26, 2019

Related U.S. Application Data
(60) Provisional application No. 62/412,946, filed on Oct. 26, 2016.

(51) Int. Cl. G01N 33/574; C07K 16/28; A61K 45/06; A61P 35/04; A61K 39/395; A61K 47/68

(52) U.S. Cl. CPC G01N 33/57492; C07K 16/2896; A61K 45/06; A61P 35/04; G01N 2333/70596; A61K 47/6855; A61K 47/6849; G01N 33/57415; A61K 39/39558

(57) ABSTRACT

The current disclosure relates to biomarkers for chemo-residual tumor cells cancer in a subject and methods of treating same.

### CROSS-REFERENCE TO RELATED APPLICATIONS

This application claims priority to U.S. Provisional Application 62/412,946 which was filed on Oct. 26, 2016, the contents of which are incorporated by reference in their entirety.

### STATEMENT REGARDING FEDERALLY SPONSORED RESEARCH

This invention was made with government support under W81XWH-13-1 awarded by the DOD Breast Cancer Research Program. The government has certain rights in the invention.

### BACKGROUND OF THE INVENTION

The field of the invention is related to diagnosis, prognosis and treatment of cancer, specifically chemotherapy resistant cancer. More particularly, the invention relates to determining and diagnosing chemo-resistant tumor cell populations and methods of treatment.

Although most triple-negative breast cancer (TNBC) patients initially respond to chemotherapy, residual tumor cells frequently persist and drive recurrent tumor growth. These residual tumor cells are thought to be responsible for recurrent tumor growth (local and distant), which frequently occurs within 3 years of treatment [1], accounting for the high mortality rate of this breast cancer subtype. The clinically unmet need for better therapeutic approaches to treat this disease underscores the importance of characterizing the signaling pathways in residual tumor cells that drive tumor recurrence post-therapy.

It is now well-appreciated that tumors are heterogeneous, being composed of chemotherapy-sensitive and chemotherapy-resistant tumor cell subpopulations [2, 3]. Because the resistant subpopulations are frequently under-represented in the tumor bulk, the identification of markers and/or behaviors of chemo-resistant subpopulations has proven elusive. Several studies indicate that chemo-resistance is associated with cancer stem-like cell behaviors [4-8]. However, the relevance of cancer stem cell-like populations to TN breast cancer recurrence remains controversial.

The present disclosure provides methods of identifying residual tumors as well as methods of treating them.

### SUMMARY OF THE INVENTION

The present disclosure provides, in part, biomarkers for identifying chemo-resistant tumor cells, such as TNBCs, and methods of treating a subject having said chemo-resistant TNBCs.

Accordingly, one aspect of the present disclosure provides a method of determining the risk of, prognosis of, and/or diagnosis of chemo-residual tumor growth in a subject following initial treatment comprising, consisting of, or consisting essentially of quantifying the amount of at least one biomarker present in a biological sample derived from the subject, wherein the biomarker is associated with chemo-residual tumor growth.

Another aspect of the present disclosure provides a method of predicting chemo-residual tumor cell growth in a subject having received treatment comprising, consisting of, or consisting essentially of: (a) obtaining a biological sample from a subject; (b) determining the expression level of one or more biomarkers that are associated with chemo-residual tumor cell growth in the biological sample; (c) comparing the expression level of the biomarker(s) in the biological sample with that of a control, wherein the presence of one or more of the biomarkers in the sample that is in an amount greater than that of the control indicates the risk of chemo-residual tumor cell growth; and (d) administering appropriate anti-cancer therapy if one or more of the biomarkers are expressed.

A method of treating a subject having chemo-residual tumor cell growth comprising, consisting of, or consisting essentially of administering to the subject a therapeutically effective amount of an antibody specific for chemo-residual tumor cells. In some aspects, the antibody is administered with a therapeutically effective amount of one or more anticancer drugs in combination to treat the subject.

In another aspect, the disclosure provides a method treating, reducing or inhibiting metastatic cancer growth in a subject, the method comprising, consisting of, or consisting essentially administering to the subject a therapeutically effective amount of an antibody specific to a biomarker for chemo-resistant tumor cells, wherein the administration reduces or inhibits metastatic cancer growth in the subject. In some aspects, the antibody is administered with a therapeutically effective amount of one or more anticancer drugs in combination with the antibody to treat, reduce or inhibit metastatic cancer growth the subject.

In yet another aspect, the disclosure provides a method of targeting a cancer therapy to chemo-resistant tumor cells within a subject, the method comprising: (a) detecting a biomarker specific for chemo-resistant tumor cells in a sample from the subject; and (b) administering an effective amount of an antibody specific to the biomarker, wherein the antibody targets the chemo-resistant tumor cells within the subject. In some aspects, the antibody is administered with a therapeutically effective amount of one or more anticancer drugs in combination.

In some embodiments, the biomarker comprises pro-N-cadherin.

In other embodiments, the tumor comprises breast cancer. In certain embodiments, the tumor comprises TNBC.

In other embodiments, the antibody comprises an antibody against pro-N-cadherin. In certain embodiments, the antibody comprises a monoclonal antibody (mAb).

In some embodiments, the subject is a mammal. In other embodiments, the subject is a human.

In other embodiments, the biological sample is selected from the group consisting of tissues, cells, biopsies, blood, lymph, serum, plasma, urine, saliva, mucus, and tears. In certain embodiments, the sample comprises biopsies.

Another aspect of the present disclosure provides all that is disclosed and illustrated herein.

The foregoing and other aspects and advantages of the invention will appear from the following description. In the description, reference is made to the accompanying drawings which form a part hereof, and in which there are shown, by way of illustration, preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

FIG. 1A shows chemo-residual triple-negative (TN) breast tumor cells emanating from short-term chemotherapy treatment model exhibit increased invasive phenotype. SUM159 and BT549 tumor cells were exposed to docetaxel (100 nM) for 2 d, after which drug was removed. On d8, only a sub-population of chemo-residual cells remained. Approximately two weeks after chemotherapy withdrawal, these cells resumed growth, establishing colonies.

FIG. 1B demonstrates the relative proliferative potential of parental and chemo-residual tumor cells (harvested on d18) was measured in a thymidine uptake assay. Results are reported as mean thymidine uptake from six wells (+/−SEM) for each cell population. Note that chemo-residual tumor cells exhibited reduced proliferation compared to parental tumor cells. **, SUM159, p=5.5×10-¹1; **, BT549, p=0.0001.

FIG. 1C demonstrates the invasive potential of parental and chemo-residual SUM159 tumor cells was measured in a Matrigel transwell assay. Top panel shows a representative field of crystal-violet stained invasive cells (100× magnification). Bottom panel shows quantitation of invasion, determined by counting the mean# invasive cells from triplicate wells [+/−standard error of the mean (SEM)] for each of the cell populations.

FIG. 1D demonstrates the invasive potential of parental and chemo-residual BT549 tumor cells was measured in a Matrigel transwell assay. Top panel shows a representative field of crystal-violet stained invasive cells (100× magnification). Bottom panel shows quantitation of invasion, determined by counting the mean# invasive cells from triplicate wells [+/−standard error of the mean (SEM)] for each of the cell populations. Similar results were obtained in at least 3 independent trials for A-D. **, SUM159; p=0.01; **, BT549-p=0.005, t-test. Similar results were obtained in at least 3 independent trials for A-D. **, SUM159; p=0.01; **, BT549-p=0.005, t-test.

FIG. 2A shows Chemo-residual TN breast tumor cells exhibit increased lung colonization. Luciferase-expressing parental and chemo-residual SUM159 cells (harvested on d18 as in FIG. 1) were injected into the tail vein of NSG mice (ten mice per group). On d33, luciferase-expressing lung colonies were visualized by luminescence (left panel). Frequency of lung colonization for each group (n=10), was assessed by luciferase signal, is indicated. *, p=0.03.

FIG. 2B show chemo-residual TN breast tumor cells exhibit increased lung colonization after 34 d. At 34 days, animals were sacrificed, and lungs were removed and photographed (left panel). Macro-metastases were counted, and are reported as median number macroscopic metastases/mouse (right panel).

FIG. 3A shows the precursor (pro) form of N-cadherin is upregulated on the cell surface of chemo-residual TN tumor cells. mRNA was isolated from parental and chemo-residual SUM159 tumor cells (harvested on d18, as in FIG. 1A). N-cadherin and beta actin levels were determined by quantitative real time PCR. Data are reported as the ratio of N-cadherin/beta actin (+/−SD from three trials). *, p=0.05, t-test.

FIG. 3B also shows the precursor form of N-cadherin chemo-residual tumor cells. Total cell extracts were obtained from EDTA-detached parental and chemo-residual SUM159 tumor cells. Equivalent amounts were immunoblotted with an N-cadherin antibody, followed by the appropriate IRdye-labelled secondary antibody. Protein bands were detected by Odyssey infrared imaging. Similar results were observed in 4 independent trials. Note the presence of increased levels of a high molecular weight N-cadherin species in chemo-residual cells compared to parental cells.

FIG. 3C also shows pro-N-cadherin in chemo-residual tumor cells. Total cell extracts were obtained from SUM159 and BT549 parental and chemo-residual tumor cells described in FIG. 1. In the top panels, equivalent amounts of protein were immunoblotted with pro-N-cadherin, N-cadherin, or Tubulin antibody, followed by IRDye conjugated secondary antibody. Similar results were obtained in three independent experiments. Bottom panels show the ratio of Pro-N-cadherin to Tubulin from three independent trials. SUM159*, p=0.05, t-test. BT549*, p=0.03.

FIG. 3D shows Pro-N-cadherin expression in chemo-residual SUM159 tumor cells. Chemo-residual SUM159 tumor cells emanating from our model were subjected to a second round of short-term docetaxel (100 nM) treatment using the same methods as described in FIG. 1. Pro-N-cadherin expression levels in chemo-resistant tumor cells generated after one or two rounds of docetaxel treatment were assessed as described in C.

FIG. 3E also shows level of Pro-N-cadherin pre and post chemotherapy. Parental and chemo-residual SUM159 tumor cells (generated in FIG. 1A) were harvested with EDTA (+/−SD), stained with a pro-N-cadherin or N-cadherin antibody, followed by FITC-conjugated secondary antibody, and analyzed by flow cytometry. Histograms are shown in the left panel. Intensity of staining is indicated as mean channel fluorescence in the right panel. Similar results were obtained in three independent trials.

FIG. 4A shows a sub-population of TN tumor cells expressing cell surface pro-N-cadherin exhibits increased invasive behavior. SUM159 cells were stained with a Pro-N-cadherin antibody (faint line) or an isotype control antibody (bold line). Pro-N-cadherin-positive (M2) and pro-N-cadherin-negative (M1) SUM159 tumor cells were isolated by cell sorting.

FIG. 4B also shows the invasive behavior of pro-N-cadherin expressing cells. Invasive potential of pro-N-cadherin-sorted TN tumor cell subpopulations was determined in matrigel-coated transwells as in FIGS. 1C and 1D. Top panel shows a representative field of crystal violet stained invasive cells (100× magnification). Bottom panel shows quantitation of invasion, determined by counting the mean# invasive cells from triplicate wells (+/−SEM). Similar results were obtained in two independent trials. *, p=0.01, t-test.

FIG. 4C shows invasive behavior of pro-N-cadherin expressing cells pre and post chemotherapy. Parental and chemo-residual SUM159 cells were placed in matrigel-coated Transwell chambers for 4 h+/−monoclonal antibody specific for the N-cadherin precursor domain (Pro domain mAb; 10A10)(30] or isotype control antibody (lgG1) at a concentration of 5 μg/ml. Mean# invasive cells from triplicate wells (+/−SEM) was determined. Similar results were obtained in three independent trials. *, p=0.03, **p=0.007.

FIG. 4D shows survival of tumor cells that are positive or negative for Pro-N cadherin after chemotherapy. Pro-N-cadherin-positive and pro-N-cadherin-negative SUM159 sorted cells were exposed to the indicated docetaxel concentrations and surviving fraction was determined in a clonogenic assay. Mean# colonies from three wells (+/−SEM) was determined for each cell population. The t-test was implemented to determine statistically significantly differences in surviving fraction for the two sorted populations at each docetaxel concentration. *50 nM, p=0.04; *75 nM, p=0.02; *100 nM, p=0.01; *150 nM, p=0.01.

FIG. 5 shows in vitro luminescence of parental and chemo-residual SUM159 tumor cells. Relative luminescence was determined in equal numbers of parental and chemo-residual tumor cells. Note that chemo-residual cells exhibit reduced luminescence compared to parental cells. Similar results were obtained in 4 independent trials.

FIG. 6A demonstrates chemo-residual TN breast tumor cells derived from a short-term chemotherapy treatment model do not exhibit increased cancer stem-like/tumor initiating activities compared to parental tumor cells. Parental and chemo-residual SUM159 cells (harvested on d18 as in FIG. 1) were seeded at equal numbers into a non-adherent mammosphere assay. Number of spheres (>50 μm) was counted after 7 d using Gel Count. Data are reported as number of spheres from 3 wells (+/−SEM) (left panel). Blank well contained no added cells. *, p=0.03, t-test. Representative fields for spheres generated from parental and chemo-residual SUM159 tumor cells are shown in the right panel. Similar results were obtained in 3 independent trials.

FIG. 6B also demonstrates chemo-residual TN breast tumor cells derived from a short-term chemotherapy treatment model do not exhibit increased cancer stem-like/tumor initiating activities compared to parental tumor cells. Cells from primary spheres generated in A were trypsinized into single cells and seeded at equal numbers into a secondary sphere assay. Spheres were counted as in A. Representative sphere fields are shown in the right panel. **, p=0.01, t-test.

FIG. 6C shows chemo-resistant tumor cells from our model did not exhibit an increased ability to grow as non-adherent spheres. Parental (blue) and chemo-residual (red) SUM159 cells were injected into the inguinal mammary gland of NSG mice in a dilution series (10\hat{ }5, 10\hat{ }4, 10\hat{ }3, 10\hat{ }2) (10 mice/group). Mice were monitored for breast tumor growth three times per week. Palpable tumors were measured with calipers. Data is plotted as percent of tumors smaller than 750 mm3 over time. P values, as determined by the log-rank Mantel-Cox test, are indicated. Note that no difference in tumor take was observed between parental and chemo-residual TN tumor cells for any cell injection number.

FIG. 7A shows tumor growth rate of parental and chemo-residual SUM159 tumor cells. Tumor growth rate in mice receiving a graft of 105 SUM159 parental (blue) or SUM159 chemo-residual (red) tumor cells/mouse.

FIG. 7B shows tumor growth rate of parental and chemo-residual SUM159 tumor cells. Tumor growth rate in mice receiving a graft of 104 SUM159 parental (blue) or SUM159 chemo-residual (red) tumor cells/mouse.

FIG. 7C shows tumor growth rate of parental and chemo-residual SUM159 tumor cells. Tumor growth rate in mice receiving a graft of 103 SUM159 parental (blue) or SUM159 chemo-residual (red) tumor cells/mouse.

FIG. 7D shows tumor growth rate of parental and chemo-residual SUM159 tumor cells. Tumor growth rate in mice receiving a graft of 102 SUM159 parental (blue) or SUM159 chemo-residual (red) tumor cells/mouse.

FIG. 8 shows pro-N-cadherin immunohistochemistry on pre- and post-neoadjuvant chemotherapy-treated TN breast cancer cases. Matched cases were obtained from six TNBC patients pre- and post-neoadjuvant chemotherapy treatment. A representative matched case is shown. Note that nuclear/peri-nuclear staining (white arrows) is observed both pre- and post-chemotherapy. However, cell surface pro-N-cadherin staining (black arrows) is only observed post-chemotherapy treatment.

FIG. 9A demonstrates cytotoxicity of pro-N-cadherin antibody in PC3 cells. Human mCRPC cells (PC3) were incubated with isotype control IgG (control) or pro-N-cadherin mAb (10A10) in triplicate wells. After 24 hours, cells were harvested and stained with trypan blue. % trypan blue(+) cells (+/−SD) is shown)(***p<0.001).

FIG. 9B demonstrates the ability of Pro-N-cadherin antibody (10A10) to reduce the number of viable PC3 cells. PC3 cells were incubated with control IgG (control) or pro-N-cadherin mAb at the indicated concentrations in triplicate wells. After 24 hours, cells were harvested, and number of live cells (+/−SD); p<0.05) was determined by trypan blue staining.

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

Unless otherwise defined, all technical terms used herein have the same meaning as commonly understood by one of ordinary skill in the art to which this disclosure belongs.

The present disclosure provides, in part, biomarkers for determining if a cancer is metastatic. The present disclosure provides, in part, biomarkers for determining whether a cancer is metastatic, for example breast or prostate cancer.

The present disclosure provides, in another part, biomarkers for determining if a cancer is chemo-resistant. Specifically, in one embodiment, biomarkers for chemo-resistant breast cancer or prostate cancer.

### Definitions

As used herein, the term “biomarker” refers to a naturally occurring biological molecule present in a subject at varying concentrations useful in predicting the risk or incidence of a disease or a condition, such as chemo-residual tumor cell growth. For example, the biomarker can be a protein present in higher or lower amounts in a subject at risk for chemo-residual tumor cell growth. The biomarker can include nucleic acids, ribonucleic acids, or a polypeptide used as an indicator or marker for chemo-residual tumor growth in the subject. In some embodiments, the biomarker is a protein. A biomarker may also comprise any naturally or nonnaturally occurring polymorphism (e.g., single-nucleotide polymorphism [SNP]) present in a subject that is useful in predicting the risk or incidence of metastatic cancer such as metastatic breast cancer. In certain embodiments, the biomarker comprises pro-N-cadherin.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In certain embodiments, the treatment comprises anti-cancer therapy and/or treatments. The term “treatment” can be characterized by one or more of the following: (a) the reducing, slowing or inhibiting the growth of cancer and cancer cells, including slowing or inhibiting the growth of chemo-resistant or chemo-residual tumor cells; (b) preventing the further growth of tumors; (c) reducing or preventing the metastasis of cancer cells within a patient; (d) reducing or ameliorating at least one symptom of cancer. In some embodiments, the optimum effective amount can be readily determined by one skilled in the art using routine experimentation.

The terms “chemo-residual” and “chemo-resistant” with regard to tumor and tumor cells are used interchangeably. These chemo-residual or chemo-resistant tumor cells are cells that survive one or more rounds of treatment with a chemotherapeutic agent.

The term “effective amount” or “therapeutically effective amount” refers to an amount sufficient to effect beneficial or desirable biological and/or clinical results. That result can be reducing, inhibiting or preventing the growth of cancer cells, reducing, inhibiting or preventing metastasis of the cancer cells or invasiveness of the cancer cells or metastasis, or reducing, alleviating, inhibiting or preventing one or more symptoms of the cancer or metastasis thereof, or any other desired alteration of a biological system. An “effective treatment” refers to treatment producing a beneficial effect, e.g., amelioration of at least one symptom of a cancer. A beneficial effect can take the form of an improvement over baseline, i.e., an improvement over a measurement or observation made prior to initiation of therapy according to the method. A beneficial effect can also take the form of reducing, inhibiting or preventing further growth of cancer cells, reducing, inhibiting or preventing metastasis of the cancer cells or invasiveness of the cancer cells or metastasis or reducing, alleviating, inhibiting or preventing one or more symptoms of the cancer or metastasis thereof. Such effective treatment may, e.g., reduce patient pain, reduce the size or number of cancer cells, may reduce or prevent metastasis of a cancer cell, or may slow cancer or metastatic cell growth.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient is suffering from, or at risk of developing chemo-residual tumor cell growth.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In one embodiment, the biological sample is a biopsy (such as a tumor biopsy). A biological sample may be obtained directly from a subject (e.g., by blood or tissue sampling) or from a third party (e.g., received from an intermediary, such as a healthcare provider or lab technician).

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

As is known in the art, a cancer is generally considered as uncontrolled cell growth. The methods of the present invention can be used to treat any cancer, any metastases thereof, and any chemo-residual growth thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma. Specifically, cancers in which pro-N-cadherin are expressed on the cancer cell surface are contemplated.

In a preferred embodiment, the cancer comprises breast cancer. In a more preferred embodiments, the cancer comprises TNBC. In another embodiment, the cancer is prostate cancer.

The terms “metastasis” or “secondary tumor” refer to cancer cells that have spread to a secondary site, e.g., outside of the original primary cancer site. Secondary sites include, but are not limited to, for example, the lymphatic system, skin, distant organs (e.g., liver, stomach, pancreas, brain, etc.) and the like and will differ depending on the site of the primary tumor.

The present disclosure provides, in part, biomarkers for identifying chemo-resistant tumor cells, such as TNBCs, and methods of treating a subject having said chemo-resistant tumor cells, such as TNBCs.

Accordingly, one aspect of the present disclosure provides a method of determining the risk of, prognosis of, and/or diagnosis of chemo-residual tumor growth in a subject following initial treatment comprising, consisting of, or consisting essentially of quantifying the amount of at least one biomarker present in a biological sample derived from the subject, wherein the biomarker is associated with chemo-residual tumor growth.

The term “initial treatment” refers to the first treatment administered to a subject in order to treat or combat the cancer. The initial treatment may be chemotherapy.

In some embodiments, this method can be done before initial treatment in order to determine the risk of, prognosis of and or diagnosis of chemo-residual tumor growth in a subject. In such an embodiment, the method of determining the risk of, prognosis of, and/or diagnosis of chemo-residual tumor growth in a subject comprising, consisting of, or consisting essentially of quantifying the amount of at least one biomarker present in a biological sample derived from the subject, wherein the biomarker is associated with chemo-residual tumor growth.

In the instances in which chemo-resistant tumor cells are indicated as present by a positive signal for one or more biomarkers, the subject can subsequently be treated by administering an antibody to the biomarker. The present inventors have found that the antibody specific to the biomarker pro-N-cadherein is able to kill the chemo-resistant TNBC cells. No other therapies up until this point have been able to kill TNBC cells. Further, it is contemplated that the antibody to the biomarker (e.g. pro-N-cadherin antibody) can be used in combination with an anti-cancer therapy, e.g. chemotherapy in order to treat the cancer. Not to be bound by any theory, but it is believe that the combination of the antibody which targets the chemo-resistant tumor cells can be used in combination with the anti-cancer treatment (e.g. chemotherapy) which can target the non-chemo-resistant tumor cells in order to treat the entire cancer or tumor (e.g. chemo-resistant and chemo-sensitive tumor cells).

In some embodiments, the antibody specific to the chemo-resistant tumor cells (e.g. pro-N-cadherein) is used in combination with one or more anti-cancer therapies or drugs in order to reduce or inhibit the chemo-resistant tumor cell growth within the subject. In such an embodiment, the initial treatment may be enhanced in order to reduce the amount of chemo-resistant tumor cells within the subject upon initial treatment and or subsequent treatments. The detection of one or biomarkers within the sample of a subject allows for the determination of the appropriate use of the antibody to the biomarker (e.g. an anti-pro-N-cadherin antibody) in order to enhance the anti-cancer treatment and reduce, inhibit or kill the chemo-resistant tumor cells. Thus, in some embodiments the antibody is used to kill chemo-resistant cell lines in combination with an anti-cancer treatment that is able to kill chemo-sensitive tumor cells.

Another aspect of the present disclosure provides a method of predicting chemo-residual tumor cell growth in a subject having received treatment comprising, consisting of, or consisting essentially of: (a) obtaining a biological sample from a subject; (b) determining the expression level of one or more biomarkers that are associated with chemo-residual tumor cell growth in the biological sample; (c) comparing the expression level of the biomarker(s) in the biological sample with that of a control, wherein the presence of one or more of the biomarkers in the sample that is in an amount greater than that of the control indicates the risk of chemo-residual tumor cell growth; and (d) administering appropriate anti-cancer therapy if one or more of the biomarkers are expressed. IN a preferred embodiment, the appropriate anti-cancer therapy is a pro-N-cadherin antibody, wherein the biomarker for the chemo-resistant cells is pro-N-cadherin.

In some embodiments, the biomarker comprises pro-N-cadherin.

Yet another aspect of the present disclosure provides a method of treating a subject having chemo-residual tumor cell growth comprising, consisting of, or consisting essentially of administering to the subject a therapeutically effective amount of an antibody specific for the associated with chemo-residual tumor cell growth.

In some embodiments, the antibody can be administered in combination with an anti-cancer therapy or drug.

As used herein, the term “anti-cancer therapy” or “anticancer drugs” refers to any drug or therapy that can be used to treat cancer. Such drugs/therapies include, but are not limited to, chemotherapy agents/therapies, cytotoxic agents/therapies, antibiotics/therapies, chemotherapy protein synthesis inhibitors/therapies, CDK4/6 inhibitors/therapies, and the like. It should be understood that these therapies can be administered to a subject alone or in combination and are dependent on many variables, such as the type of cancer, aggressiveness of the cancer, patient specifics and the like and such determination can be readily determined by one skilled in the art.

As used herein the term “chemotherapy” or “chemotherapeutic agent” refers to treatment with a cytostatic or cytotoxic agent (i.e., a compound) to reduce or eliminate the growth or proliferation of undesirable cells, for example cancer cells. Thus, as used herein, “chemotherapy” or “chemotherapeutic agent” refers to a cytotoxic or cytostatic agent used to treat a proliferative disorder, for example cancer. The cytotoxic effect of the agent can be, but is not required to be, the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect.

Thus, a “cytotoxic agent” can be any one or any combination of compounds also described as “antineoplastic” agents or “chemotherapeutic agents.” Such compounds include, but are not limited to, DNA damaging compounds and other chemicals that can kill cells. “DNA damaging chemotherapeutic agents” include, but are not limited to, alkylating agents, DNA intercalators, protein synthesis inhibitors, inhibitors of DNA or RNA synthesis, DNA base analogs, topoisomerase inhibitors, and telomerase inhibitors or telomeric DNA binding compounds. For example, alkylating agents include alkyl sulfonates, such as busulfan, improsulfan, and piposulfan; aziridines, such as a benzodizepa, carboquone, meturedepa, and uredepa; ethylenimines and methylmelamines, such as altretamine, triethylenemelamine, triethylenephosphoramide, triethylenethiophosphoramide, and trimethylolmelamine; nitrogen mustards such as chlorambucil, chlornaphazine, cydophosphamide, estramustine, iphosphamide, mechlorethamine, mechlorethamine oxide hydrochloride, melphalan, novembichine, phenesterine, prednimustine, trofosfamide, and uracil mustard; and nitroso ureas, such as carmustine, chlorozotocin, fotemustine, lomustine, nimustine, and ranimustine.

The anticancer agents may also be, in some embodiments, CDK4/6 inhibitors. Suitable CD4/6 inhibitors are known in the art and include, but are not limited to, Ribocidib, Palbociclib (PD-0332991) (inhibitor of CDK4 and CDK6) Abemaciclib (LY2835219) (trade name Verzenio) acts as a selective inhibitor for CDK4 and CDK6.

In some embodiments, the anti-cancer therapy is an antibiotic. Antibiotics used in the treatment of cancer include, but are not limited to, for example, dactinomycin, daunorubicin, doxorubicin, idarubicin, bleomycin sulfate, mytomycin, plicamycin, and streptozocin. Chemotherapeutic antimetabolites include mercaptopurine, thioguanine, cladribine, fludarabine phosphate, fluorouracil (5-FU), floxuridine, cytarabine, pentostatin, methotrexate, and azathioprine, acyclovir, adenine .beta.-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycytidine, cytosine beta.-1-D-arabinoside, diazooxynorleucine, dideoxynudeosides, 5-fluorodeoxycytidine, 5-fluorodeoxyuridine, and hydroxyurea.

In some embodiments, the anti-cancer therapy is a chemotherapeutic protein synthesis inhibitor, a DNA synthesis inhibitor, a topoisomerase inhibitor, and the like. Chemotherapeutic protein synthesis inhibitors include, but are not limited to, abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine. Additional protein synthesis inhibitors include modeccin, neomycin, norvaline, pactamycin, paromomycine, puromycin, ricin, shiga toxin, showdomycin, sparsomycin, spectinomycin, streptomycin, tetracycline, thiostrepton, and trimethoprim. Inhibitors of DNA synthesis, include, but are not limited to, for example, alkylating agents such as dimethyl sulfate, mitomycin C, nitrogen and sulfur mustards; intercalating agents, such as acridine dyes, actinomycins, adriamycin, anthracenes, benzopyrene, ethidium bromide, propidium diiodide-intertwining; and other agents, such as distamycin and netropsin. Topoisomerase inhibitors, such as coumermycin, nalidixic acid, novobiocin, and oxolinic acid; inhibitors of cell division, including colcemide, colchicine, vinblastine, and vincristine; and RNA synthesis inhibitors including actinomycin D, alpha-amanitine and other fungal amatoxins, cordycepin (3′-deoxyadenosine), dichlororibofuranosyl benzimidazole, rifampicine, streptovaricin, and streptolydigin also can be used as the DNA damaging compound.

Further chemotherapeutic agents that can be used in the present invention include, but are not limited to, adrimycin, 5-fluorouracil (5FU), 6-mercaptopurine, gemcitabine, melphalan, chlorambucil, mitomycin, irinotecan, mitoxantrone, etoposide, camptothecin, actinomycin-D, mitomycin, cisplatin, hydrogen peroxide, carboplatin, procarbazine, mechlorethamine, cydophosphamide, ifosfamide, melphalan, chlorambucil, busulfan, nitrosurea, dactinomycin, daunorubicin, doxorubicin, bleomycin, plicomycin, tamoxifen, taxol, transplatinum, vinblastine, vinblastin, carmustine, cytarabine, mechlorethamine, chlorambucil, streptozocin, lomustine, temozolomide, thiotepa, altretamine, oxaliplatin, campothecin, and methotrexate, and the like, and similar acting-type agents. In one embodiment, the DNA damaging chemotherapeutic agent is selected from the group consisting of cisplatin, carboplatin, campothecin, doxorubicin, and etoposide.

In certain alternative embodiments, the antibody to pro-N-cadherin can be used in combination with a chemotherapeutic to treat cancer or proliferative disorder. The antibody described herein may provide an additive or synergistic effect to the chemotherapeutic, resulting in a greater anti-cancer effect than seen with the use of the chemotherapeutic alone, specifically targeting the chemo-resistant tumor cells. In other words, the antibody used in combination with an anti-cancer therapy will allow for the targeting of both chemo-resistant and chemo-sensitive cells within the cancer (by use of the antibody targeting chemo-resistant and the anti-cancer therapy targeting chemo-sensitive cells), thereby resulting in an increased reduction in tumor cells or tumor volume by targeting the different cells within the tumor.

In one embodiment, the antibody described herein can be combined with one or more of the chemotherapeutic compounds described above. In one embodiment, the antibody to the biomarker, e.g. pro-N-cadherin, can be combined with a chemotherapeutic selected from, but not limited to, for example, tamoxifen, midazolam, letrozole, bortezomib, anastrozole, goserelin, an mTOR inhibitor, a PI3 kinase inhibitors, dual mTOR-PI3K inhibitors, MEK inhibitors, RAS inhibitors, ALK inhibitors, HSP inhibitors (for example, HSP70 and HSP 90 inhibitors, or a combination thereof), BCL-2 inhibitors, apopototic inducing compounds, AKT inhibitors, including but not limited to, MK-2206, GSK690693, Perifosine, (KRX-0401), GDC-0068, Triciribine, AZD5363, Honokiol, PF-04691502, and Miltefosine, PD-1 inhibitors including but not limited to, Nivolumab, CT-011, MK-3475, BMS936558, and AMP-514 or FLT-3 inhibitors, including but not limited to, P406, Dovitinib, Quizartinib (AC220), Amuvatinib (MP-470), Tandutinib (MLN518), ENMD-2076, and KW-2449, or combinations thereof. Examples of mTOR inhibitors include but are not limited to rapamycin and its analogs, everolimus (Afinitor), temsirolimus, ridaforolimus, sirolimus, and deforolimus. Examples of P13 kinase inhibitors include but are not limited to Wortmannin, demethoxyviridin, perifosine, idelalisib, PX-866, IPI-145 (Infinity), BAY 80-6946, BEZ235, RP6503, TGR 1202 (RP5264), MLN1117 (INK1117), Pictilisib, Buparlisib, SAR245408 (XL147), SAR245409 (XL765), Palomid 529, ZSTK474, PWT33597, RP6530, CUDC-907, and AEZS-136. Examples of MEK inhibitors include but are not limited to Tametinib, Selumetinib, MEK162, GDC-0973 (XL518), and PD0325901. Examples of RAS inhibitors include but are not limited to Reolysin and siG12D LODER. Examples of ALK inhibitors include but are not limited to Crizotinib, AP26113, and LDK378. HSP inhibitors include but are not limited to Geldanamycin or 17-N-Allylamino-17-demethoxygeldanamycin (17AAG), and Radicicol.

In one embodiment, the pro-N-cadherin antibody can be combined with a chemotherapeutic selected from, but are not limited to, Imatinib mesylate (Gleevac®), Dasatinib (Sprycel®), Nilotinib (Tasigna®), Bosutinib (Bosulif®), Trastuzumab (Herceptin®), Pertuzumab (Perjeta™), Lapatinib (Tykerb®), Gefitinib (Iressa®), Erlotinib (Tarceva®), Cetuximab (Erbitux®), Panitumumab (Vectibix®), Vandetanib (Caprelsa®), Vemurafenib (Zelboraf®), Vorinostat (Zolinza®), Romidepsin (Istodax®), Bexarotene (Tagretin®), Alitretinoin (Panretin®), Tretinoin (Vesanoid®), Carfilizomib (Kyprolis™), Pralatrexate (Folotyn®), Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™).

In some embodiments, the antibody may further be linked to the anti-cancer drug or therapy. In one embodiment, the antibody is conjugated to any one of the anti-cancer therapies described herein. In one embodiment, the antibody can be conjugated to a CDK4/6 inhibitor or another inhibitor described herein.

In other embodiments, the antibody comprises an antibody against pro-N-cadherin. In certain embodiments, the antibody comprises a monoclonal antibody (mAb).

Suitable pro-N-cadherin antibodies are antibodies that are able to bind specifically to pro-N-cadherin. In other words, they are antibodies that specifically bind to the N-terminal pro-N-cadherin region of N-cadherin that is cleaved to form N-cadherin. Suitable antibodies may bind the polypeptide sequence of proregion of N-cadherein, (e.g. MCRIAGALRTLLPLLAALLQASVEASGEIALCKTGFPEDVYSAVLSKDVHEGQPLLNVKFSNCN GKRKVQYESSEPADFKVDEDGMVYAVRSFPLSSEHAKFLIYAQDKETQEKWQVAVKLSLKPTL TEESVKESAEVEEIVFPRQFSKHSGHLQRQKR (SEQ ID NO:1) taken from sequence of human N-cadherin, (GenBank™ accession NM_001792)). The antibody may bind an epitope of pro-N-cadherin that is linear or bind to a secondary structure formed in the pro-N-cadherin domain. The antibodies contemplated for use in the invention are antibodies specific to the pro-N-caherin peptide (N-cadherin propeptide) and do not bind to the mature form of N-cadherin. As shown herein, pro-N-cadherin is specifically found on chemo-resistant tumor cells. As such, the use of an antibody specific to pro-N-cadherin allows for the specific targeting and killing of tumor cells in contrast to an antibody that binds to the mature form of N-cadherin, which is found on other normal cell types (e.g. heart). Thus, the present invention, in one embodiment, provides an targeted therapy, e.g. antibody therapy against pro-N-cadherin that allows for the specific targeting of chemo-resistant tumor cells, specifically TNBC cells. One skilled in the art would be able to determine suitable antibodies for use in the present invention. Suitable examples include, but are not limited to, pro-N-cadherin antibody described in Wahl et al., (N-cadherin-catenin complexes form prior to cleavage of the proregion and transport to the plasma membrane. J Biol Chem. 2003; 278(19):17269-17276, incorporated by reference) and Maret et al. 2010 (Maret D, Gruzglin E, Sadr M S, et al. Surface Expression of Precursor N-cadherin Promotes Tumor Cell Invasion. Neoplasia (New York, N.Y.). 2010; 12(12):1066-1080, incorporated by reference in its entirety). Further a pro-N-cadherin antibody is commercially available from R&D systems (human N-cadherin propeptide antibody, available from Bio-Techne Corporation, Minneapolis, Minn.). The antibodies contemplated herein are do not bind to the mature form of N-cadherin, therefore reducing non-specific cell targeting within a patient (i.e. do not bind to normal cells that express N-cadherin but not pro-N-cadherin).

The antibodies specific to pro-N-cadherin include whole antibodies (e.g., IgG, IgA, IgE, IgM, or IgD), monoclonal antibodies, polyclonal antibodies, and chimeric antibodies, humanized antibodies, and antibody fragments, including single chain variable fragments (ScFv), single domain antibody, and antigen-binding fragments, among others. In a preferred embodiment, the antibody is a monoclonal antibody.

In some embodiments, the anti-cancer drug/therapy is administered before the antibody. In other embodiments, the antibody is administered before the anti-cancer drug/therapy. In yet other embodiments, the antibody and anti-cancer drug/therapy are administered concurrently.

In some embodiments, the anti-cancer drug/therapy is covalently or non-covalently linked or attached to the antibody in order to target the chemo-therapeutic resistant cancer cells. For example, a complex may be made using one or more anti-cancer drugs/therapies and the pro-N-cadherin antibody to specifically target the chemo-residual tumor cells during treatment.

Pro-N-cadherin antibodies may be provided in combination with liposomes, nanoparticles or other analogous carriers loaded with an anti-cancer drug/therapy. Methods of preparing such compositions are known in the field (see, for example, Sugano et al., Antibody Targeting of Doxorubicin-loaded Liposomes Suppresses the Growth and Metastatic Spread of Established Human Lung Tumor Xenografts in Severe Combined Immunodeficient Mice, Cancer Research 60, 6942-6949, Dec. 15, 2000 and Martin et al., Nanomaterials in Analytical Chemistry, Analytical Chemistry News & Features, May 1, 1998; pp. 322 A-327 A). As used herein, the phrase “antibody in combination with an anti-cancer drug” shall not be limited by the method of manufacture and such compositions may be produced by, but not limited to, techniques of conjugating, linking, coupling and decorating known in the art.

One may wish to express the antibody as a fusion protein with a pharmacologically or therapeutically relevant peptide that acts as an additional an anti-cancer drug/therapy as the antibody itself to pro-N-caherin has been shown to kill tumor cells. Standard molecular biology techniques (e.g., restriction enzyme based subcloning or homology based subcloning) could be used to place the DNA sequence encoding a protein therapeutic in frame with the targeting antibody (usually a protein linker is also added to avoid steric hindrance). The fusion protein is then produced as one peptide in a host cell (e.g., yeast, bacteria, insect, or mammalian cell) and purified before use. Note the therapeutic does not need to be a whole protein. (For example, it can be a single peptide chain as a subunit in a protein with more than one peptide. The other peptides can be co-expressed with the vector fusion and allowed to associate in the host cell or after secretion).

In some embodiments of the present invention, antibodies may be administered with or without the above modifications. One may wish to administer the antibodies of the present invention without the modifications described above. For example, one may administer the antibodies through an intravenous injection or through intra-peritoneal and subcutaneous methods.

In other embodiments, the tumor comprises breast cancer. In certain embodiments, the tumor comprises TNBC.

In some embodiments, the subject is a mammal. In other embodiments, the subject is a human.

In other embodiments, the biological sample is selected from the group consisting of tissues, cells, biopsies, blood, lymph, serum, plasma, urine, saliva, mucus, and tears. In certain embodiments, the sample comprises biopsies.

Another aspect of the present disclosure provides all that is disclosed and illustrated herein.

Sample

The present disclosure provides a method of determining the risk of, prognosis of, and/or diagnosis of a condition such as metastatic cancer, for example metastatic breast cancer, on at least one sample obtained from a subject. In one embodiment, the subject is any mammal, but is preferably a human. The method comprises detecting and/or measuring the amount of at least one biomarker within the sample, wherein the biomarker is associated with the risk of, prognosis of, and/or diagnosis of the condition.

The present disclosure may involve obtaining more than one sample, such as two samples, such as three samples, four samples or more from subjects, and preferably the same subject. This allows the relative comparison of expression both in the presence or absence of at least biomarker (e.g. one nucleic acid) and/or the level of expression of the at least biomarker (e.g. one nucleic acid) between the two samples. Alternatively, a single sample may be compared against a “standardized” sample, such a sample comprising material or data from several samples, preferably also from several subjects.

Sample Preparation

Before analyzing the sample, it will often be desirable to perform one or more sample preparation operations upon the sample. Typically, these sample preparation operations will include such manipulations as concentration, suspension, extraction of intracellular material.

Any method required for the processing of a sample prior to detection by any of the methods noted herein falls within the scope of the present disclosure. These methods are typically well known by a person skilled in the art.

Detection

It is within the general scope of the present disclosure to provide methods for the detection of protein biomarker. An aspect of the present disclosure relates to the detection of the proteins as described in the plots and graphs of the figures contained herein. The present invention detects the protein of the pro-N-cadherin using a method that specifically detects the protein pro-N-cadherin and does not detect the processed or mature form of N-cadherin (e.g. the processed protein missing the pro-N-cadherin region).

As used herein, the term “detect” or “determine the presence of” refers to the qualitative measurement of undetectable, low, normal, or high concentrations of one or more biomarkers such as, for example, polypeptides of the pro-N-cadherin. Detection may include 1) detection in the sense of presence versus absence of one or more biomarkers as well as 2) the registration/quantification of the level or degree of expression of one or more biomarkers, depending on the method of detection employed. The term “quantify” or “quantification” may be used interchangeable, and refer to a process of determining the quantity or abundance of a substance in a sample (e.g., a biomarker), whether relative or absolute. For example, quantification may be determined by methods including but not limited to, any method able to detect proteins for example, immunohistochemistry, flow cytometry, band intensity on a Western blot, or by various other methods known in the art.

The detection of one or more biomarker molecules allows for the classification, diagnosis and prognosis of a condition such as metastatic cancer, preferably breast cancer. The classification of such conditions is of relevance both medically and scientifically and may provide important information useful for the diagnosis, prognosis and treatment of the condition. The diagnosis of a condition such as metastatic breast cancer is the affirmation of the presence of the condition, as is the object of the present disclosure, on the expression of at least one biomarker herein. Prognosis is the estimate or prediction of the probable outcome of a condition such as metastatic breast cancer and the prognosis of such is greatly facilitated by increasing the amount of information on the particular condition. The method of detection is thus a central aspect of the present disclosure.

Any method of detection falls within the general scope of the present disclosure. The detection methods may be generic for the detection of polypeptides and the like. The detection methods may be directed towards the scoring of a presence or absence of one or more biomarker molecules or may be useful in the detection of expression levels.

The detection methods can be divided into two categories herein referred to as in situ methods or screening methods. The term in situ method refers to the detection of protein molecules in a sample wherein the structure of the sample has been preserved. This may thus be a biopsy wherein the structure of the tissue is preserved. In situ methods are generally histological i.e. microscopic in nature and include but are not limited to methods such as: immunohistochemistry or any in situ methods able to detect proteins and polypeptides.

Screening methods generally employ techniques of molecular biology and most often require the preparation of the sample material in order to access the polypeptide molecules to be detected. Screening methods include, but are not limited to methods such as: flow cytometry, Western blot analysis, enzyme-linked immunosorbent assay (ELISA), and immunoelectrophoresis. Other methods understood and known by one skilled in the art for detecting proteins is contemplated for use in the present methods.

Probe

One aspect of the present disclosure is to provide a probe which can be used for the detection of a polypeptide molecule as defined herein. A probe as defined herein is a specific agent used to detect polypeptides by specifically binding to the protein, e.g. pro-N-cadherin. For example, an antibody or fragment thereof specific to pro-N-cadherin protein can be used as a probe to detect the biomarker, e.g. pro-N-cadherin in a sample. A probe may be labeled, tagged or immobilized or otherwise modified according to the requirements of the detection method chosen. A label or a tag is an entity making it possible to identify a compound to which it is associated. It is within the scope of the present disclosure to employ probes that are labeled or tagged by any means known in the art such as but not limited to: radioactive labeling, fluorescent labeling and enzymatic labeling. Furthermore the probe, labeled or not, may be immobilized to facilitate detection according to the detection method of choice and this may be accomplished according to the preferred method of the particular detection method.

The probes used may be to one or more biomarkers as disclosed herein. In a preferred embodiment, the probe is an antibody to pro-N-cadherin.

Detection Methods

Another aspect of the present disclosure regards the detection of a biomarker which is a polypeptide molecules by any method known in the art. In the following are given examples of various detection methods that can be employed for this purpose, and the present disclosure includes all the mentioned methods, but is not limited to any of these.

Immunohistochemistry

Immunohistochemistry (IHC) involves the process of selectively imaging proteins in cells of a tissue section by using antibodies binding specifically to protein. Immunohistochemical staining is widely used in the diagnosis of abnormal cells such as those found in cancerous tumors. Visualising an antibody-antigen interaction can be accomplished in a number of ways known in the art, including, but not limited to, using an antibody conjugated to an enzyme, such as peroxidase, that can catalyse a color-producing reaction (e.g. immunoperoxidase staining), an antibody tagged or conjugated with a fluorophore, such as fluorescein or rhodamine (e.g. immunofluorescence), among others.

A probe used in IHC (e.g. an antibody or fragment thereof) can be labeled with a radioactive, fluorescent or antigenic tag, so that the probe's location and quantity in the tissue can be determined using autoradiography, fluorescence microscopy or immunoassay, respectively. The sample may be any sample as herein described. The probe is likewise a probe according to any probe based upon the biomarkers mentioned herein.

Flow Cytometry

Flow cytometery can be used in the methods of detecting described herein. Flow cytometry is a laser- or impedance-based method that allows for cell counting, cell sorting, and biomarker detection by suspending cells in a stream of fluid and passing them through an electronic detection apparatus. The present methods include the use of flow cytometry to detect biomarkers on cells within samples taken from the subject. Suitable methods of flow cytometry are known in the art. In one suitable method, an antibody to pro-N-cadherin can be used in conjunction with a fluorescently tagged secondary antibody. In some embodiments, the pro-N-cadherin antibody may be directly conjugated to a fluorescence-tag. Methods of fluorescence-activated cell sorting (FACS) may also be used. It provides a method for sorting a heterogeneous mixture of biological cells into two or more containers, one cell at a time, based upon the specific light scattering and fluorescent characteristics of each cell.

Western Blot Analysis

Western blot (sometimes called the protein immunoblot) can be used in the detection methods described herein. Western blot methods are known in the art. For example, a sample may be separated by gel electrophoresis. Following electrophoretic separation, the proteins within the gel are transferred to a membrane (e.g., nitrocellulose or PVDF) on which the protein is then detected using a suitable probe, e.g. antibody specific to the biomarker. Using various methods such as staining, immunofluorescence, and radioactivity, visualization of the protein of interest can be detected on the membrane. Other suitable related techniques that can be used include, but are not limited to, dot blot analysis, and quantitative dot blot.

Enzyme-Linked Immunosorbent Assay (ELISA)

The enzyme-linked immunosorbent assay (ELISA) can also be used in the methods described herein. In some embodiments, the ELISA includes a solid-phase enzyme immunoassay (EIA) to detect the presence of a protein in a sample. ELISA also uses a probe, e.g. antibody specific to the biomarker to detect the biomarker within the sample. Suitable methods of performing ELISA are known in the art.

Immunoelectrophoresis

Immunoelectrophoresis can also be used in the methods described herein, for example, a number of biochemical methods for separation and characterization of proteins based on electrophoresis and reaction with antibodies are known in the art. The methods usually use antibodies specific to the protein to be detected.

Another aspect of the present disclosure provides all that is disclosed and illustrated herein.

Methods of Treatment

In some embodiment, the present disclosure provides methods of treating a subject having cancer. In one embodiment, the patient may have metastatic cancer. In another embodiment, the subject may have chemo-resistant cancer or the presence of chemo-resistant tumor cells.

Currently there are no targeted therapies for these chemo-resistant cells in triple-negative breast cancer patients. The present invention provides, in one embodiment a targeted therapy to triple-negative breast cancer or other metastatic cancers which express the biomarker pro-N-cadherin, the targeted therapy comprising an antibody specific to pro-N-cadherin. As demonstrated in the examples, the antibody specific to pro-N-cadherin is able to kill chemo-resistant tumor cells, specifically TNBC cells or metastatic prostate cancer cells.

The methods of treatment described herein can be used in combination with the methods of detecting, prognosing, and predicting described above. For example, in some embodiments, a method of detecting, predicting or prognosing chemo-resistant cells within a subject is combined with a subsequent treatment step comprising administering a therapeutically effective amount of an antibody to the chemo-resistant biomarker (e.g. pro-N-cadherin). In some embodiments, the antibody can be used in combination with one or more anti-cancer drugs or therapies.

In one embodiment, the invention provides a method of treating a subject having chemo-residual tumor cell growth comprising administering to the subject a therapeutically effective amount of one or more anticancer drugs and an antibody specific for a biomarker associated with chemo-residual tumor cell growth. In a preferred embodiment, the antibody comprises an antibody capable of binding to pro-N-cadherin as described herein.

In some embodiments, the method of treating a subject having chemo-residual tumor cell growth comprises: (a) obtaining a sample from the subject; and (b) detecting the presence of a biomarker within the sample, wherein the presence of the biomarker indicates the presence of chemo-residual tumor cells; and (c) administering to the subject having detected the biomarker within the subject's sample a therapeutically effective amount of an antibody to the biomarker. In some embodiments, the antibody to the biomarker can be administered in combination with one or more anti-cancer drugs or therapies. In a preferred embodiment, the biomarker is pro-N-cadherin.

Another embodiment provides a method of treating, reducing or inhibiting metastatic cancer growth in a subject, the method comprising administering to the subject a therapeutically effective amount of an antibody specific to a biomarker specific for chemo-resistant tumor cells, wherein the administration reduces or inhibits metastatic cancer growth in the subject. In some embodiments, the antibody may be used in combination with an anti-cancer drug or therapy can be administered prior to, co-currently or subsequently to administration of the antibody.

In yet another embodiment, the disclosure provides a method of targeting a cancer therapy to chemo-resistant tumor cells within a subject, the method comprising: (a) detecting a biomarker specific for chemo-resistant tumor cells in a sample from the subject; and (b) administering an effective amount of an antibody specific to the biomarker, wherein the antibody targets the chemo-resistant tumor cells within the subject. In a preferred embodiment, the biomarker is pro-N-cadherin. In another embodiment, the antibody is combined with an antic-cancer drug or therapy. In some embodiments, the anticancer drug and the antibody are administered co-currently. In one embodiment, the anticancer drug and the antibody are covalently or noncovalent linked.

In some embodiments, the present disclosure provides compositions comprising a pro-N-cadherin antibody. In another embodiment, the present disclosure provides a composition comprising a pro-N-cadherin antibody and one or more anti-cancer therapies or drugs. Suitably, the compositions are in pharmaceutically acceptable carrier, e.g. saline buffer or phosphate buffer saline. In some embodiments, the pro-N-cadherin antibody are covalently or non-covalently linked to the cancer therapy or drug. Suitable complexes can be made by one skilled in the art.

Kits

In some embodiments, kits for carrying out the methods described herein are provided. The kits provided may contain the necessary components with which to carry out one or more of the above-noted methods. In one embodiment, a kit for detecting a biomarker specific for chemo-resistant tumor cells are provided. The kit may comprise an antibody specific to the biomarker. In a preferred embodiment, the biomarker is pro-N-cadherin. In some embodiments, the detecting is by an antibody specific to the biomarker. In other embodiments, the detecting is by other methods described herein. In one embodiment, the kit comprises an antibody to pro-N-cadherin conjugated to a detection agent or magnetic beads.

In further embodiments, a control is provided. In one embodiment, the control is a positive control, for example, a sample positive for the biomarker specific for the chemo-resistant tumor cells. In another example, the control is a control obtained from a healthy individual that does not have cancer.

In further embodiments, the kits may include a composition for the treatment of a subject in which chemo-residual tumor cells have been detected. The kits may include an antibody specific to the biomarker (e.g. pro-N-cadherin antibody). In some further embodiments, the kit may further include one or more chemotherapeutic agents. In some embodiments, the antibody is directly or indirectly conjugated to the anti-cancer drug or therapy. In other embodiments, the antibody is covalently or non-covalently linked to the anti-cancer drug or therapy.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

The following examples are provided as illustration and not by way of limitation. Each publication, patent, and patent publication cited in this disclosure is incorporated in reference herein in its entirety. The present invention is not intended to be limited to the foregoing examples, but encompasses all such modifications and variations as come within the scope of the appended claims.

### EXAMPLES

### Example 1: Chemotherapy Enriches for an Invasive Triple-Negative Breast Tumor Cell Subpopulation Expressing a Precursor Form of N-Cadherin on the Cell Surface

Although most triple-negative breast cancer (TNBC) patients initially respond to chemotherapy, residual tumor cells frequently persist and drive recurrent tumor growth. Previous studies from our laboratory and others' indicate that TNBC is heterogeneous, being composed of chemo-sensitive and chemo-resistant tumor cell subpopulations. IN this example, we studied the invasive behaviors of chemo-resistant TNBC, and sought to identify markers of invasion in chemo-residual TNBC. To study the invasive behavior of TNBC tumor cells, surviving short-term chemotherapy treatment in vitro was studied using transwell invasion assays and an experimental metastasis model. mRNA expression levels of neural cadherin (N-cadherin), an adhesion molecule that promotes invasion, was assessed by PCR. Expression of N-cadherin and its precursor form (pro-N-cadherin) was assessed by immunoblotting and flow cytometry. Pro-N-cadherin immunohistochemistry was performed on tumors obtained from patients' pre- and post-neoadjuvant chemotherapy treatment.

As demonstrated in this Example, TNBC cells surviving short-term chemotherapy treatment exhibited increased invasive behavior and capacity to colonize metastatic sites compared to untreated tumor cells. The invasive behavior of chemo-resistant cells was associated with their increased cell surface expression of precursor N-cadherin (pro-N-cadherin). An antibody specific for the precursor domain of N-cadherin inhibited invasion of chemo-resistant TNBC cells. To begin to validate our findings in humans, this Example showed that the percent cell surface pro-N-cadherin (+) tumor cells increased in patients post-chemotherapy treatment.

TNBC cells surviving short-term chemotherapy treatment are more invasive than bulk tumor cells. Cell surface pro-N-cadherin expression is associated with the invasive and chemo-resistant behaviors of this tumor cell subset. The cell surface pro-N-cadherin can be used as: 1) a biomarker for TNBC recurrence and 2) a therapeutic target for eliminating chemo-residual disease.

Previously, our group described a method for studying TN breast cancer cell subpopulations enriched by short-term chemotherapy treatment[9]. In this model, short-term chemotherapy treatment of TN breast tumor cells enriches for chemo-resistant, growth-arrested tumor cells. These chemo-residual tumor cells resume growth after removing the chemotherapeutic agent, and subsequently establish drug resistant colonies [9]. This model resembles the clinical setting of a chemotherapeutic “rest period” or “drug holiday”, which occurs between chemotherapy cycles [10]. Colonies emanating from chemo-residual tumor cells after chemotherapy removal resemble recurrent tumors in that they exhibit multidrug resistance [9].

This Example shows that chemo-resistant TN breast tumor cells emanating from this short-term chemotherapy treatment model exhibit increased invasive/metastatic behavior. These results suggest that chemotherapy drives the evolution of more aggressive TN breast cancers by enriching for a highly invasive tumor cell sub-population. Moreover, we show that these chemotherapy-enriched, aggressive tumor cell subpopulations do not exhibit classic properties of cancer stem-like cells. Finally, we identify a novel adhesion marker expressed on the surface of chemo-resistant TN tumor cells that drives their invasive phenotype, and demonstrate that this marker is increased in primary TN breast cancers post neoadjuvant chemotherapy treatment.

Results:

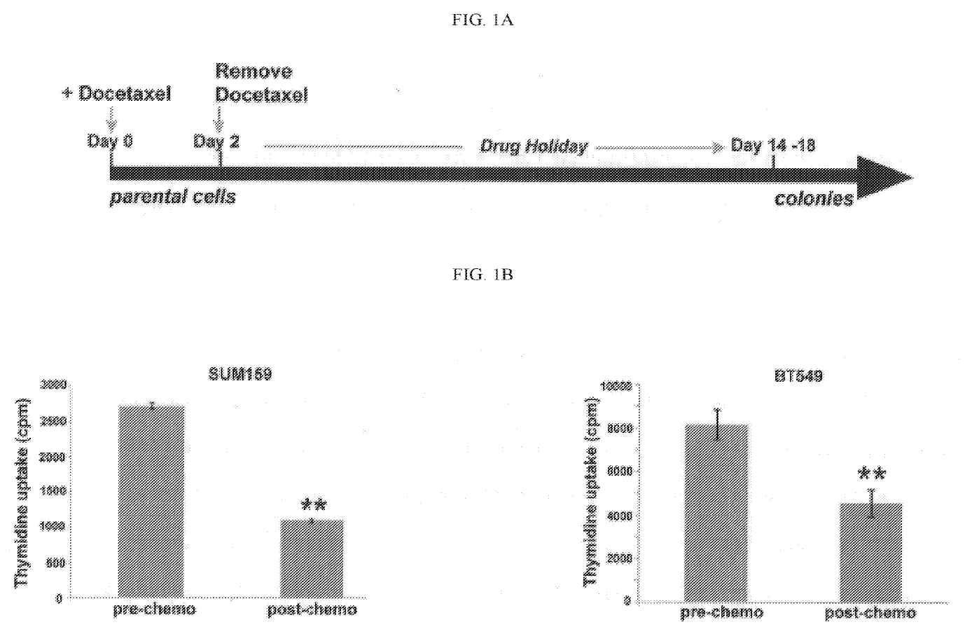

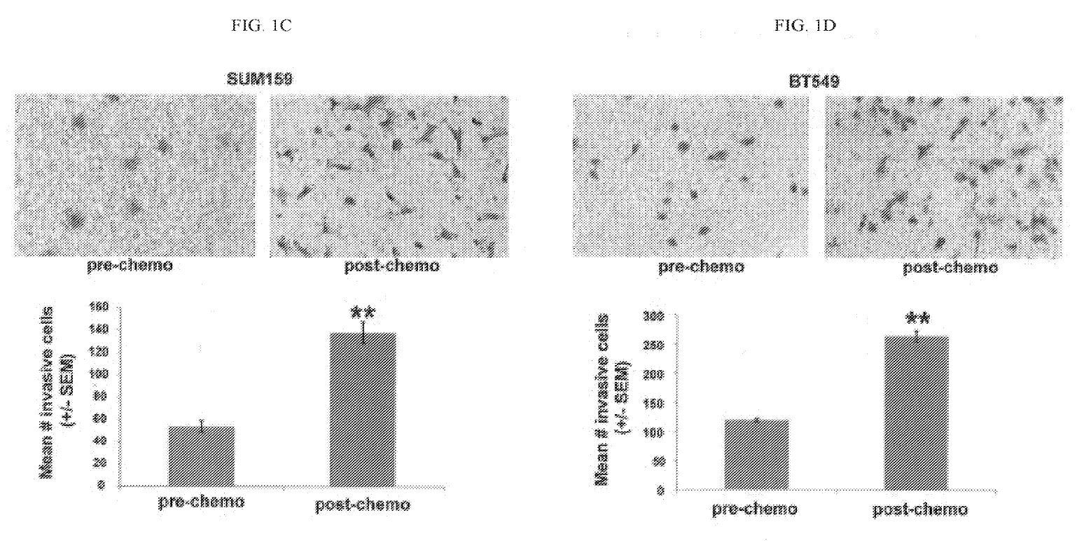

We have developed a short-term chemotherapy treatment model that enriches for a chemo-resistant subset of TN breast tumor cells [9] (FIG. 1A). In this model, triple-negative breast cancer cells (SUM159, BT549) were exposed to a clinically-relevant chemotherapy regimen (docetaxel) for two days, after which drug was removed from the medium. By day 7, we observed a subpopulation of growth-arrested tumor cells surviving chemotherapy. Approximately 2 weeks after chemotherapy removal, this chemo-residual tumor cell subpopulation resumed growth, establishing colonies (FIG. 1A). In our previous work, we showed that colonies emanating from this short-term chemotherapy-treatment model exhibit multi-drug resistance (9). In the current work, we investigated the invasive potential of chemo-resistant TN breast tumor colonies established after chemotherapy removal. As shown in FIG. 1B, colonies evolving from this short-term docetaxel treatment model exhibited reduced proliferation compared to untreated parental cells. Notably, chemo-resistant TN tumor cells arising from this model also exhibited significantly increased invasive potential, as measured in a matrigel transwell assay (FIGS. 1C&D).

Based on their increased invasive phenotype, we next sought to determine if these chemo-resistant tumor cells, when injected into the tail vein of immunocompromised mice, exhibited increased ability to colonize the lung compared to untreated tumor cells. First, luciferase-expressing SUM159 TN tumor cells were subjected to short-term docetaxel treatment as in FIG. 1A, after which chemotherapy was removed.

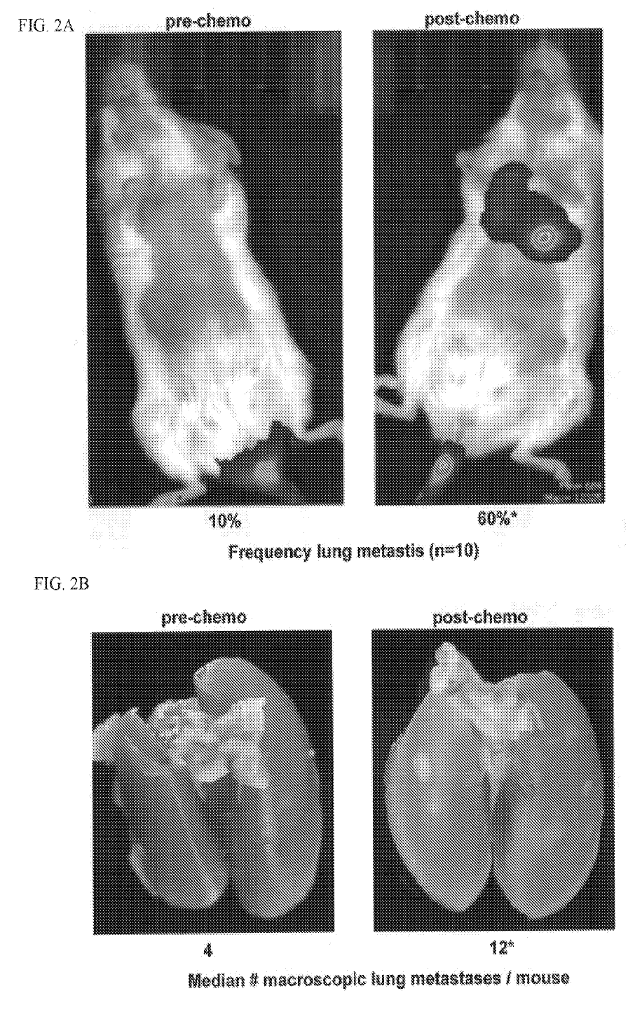

Colonies emanating from this model on day 18 were harvested, and their lung colonizing potential was measured in a tail vein injection model using NOD scid gamma (NSG) mice. Specifically, NSG mice were divided into two groups (10 mice/group). The first group was injected with luciferase-expressing parental SUM159 TN tumor cells (Pre-chemo). The second group was injected with luciferase-expressing chemo-residual SUM159 cells (Post-chemo). On day 33, the luciferase signal in the lung was determined by bioluminescence. Strikingly, TN tumor cells obtained post-chemotherapy treatment colonized the lung in six of ten mice (60%) whereas parental TN tumor cells colonized the lung in only one of ten mice (10%) (FIG. 2A).

The increased frequency of lung colonization by chemo-resistant TN tumor cells was observed despite the fact that the chemo-resistant tumor cells exhibited a lower luciferase signal/cell than parental tumor cells (FIG. 5). On day 34, animals were sacrificed, lungs were harvested, and the number of macroscopic lung metastases/mouse was determined. Lungs from mice grafted with chemo-resistant SUM159 cells emanating from our short-term chemotherapy treatment model exhibited an increased number of macroscopic lung metastases/mouse compared to those from mice grafted with parental SUM159 cells (FIG. 2B).

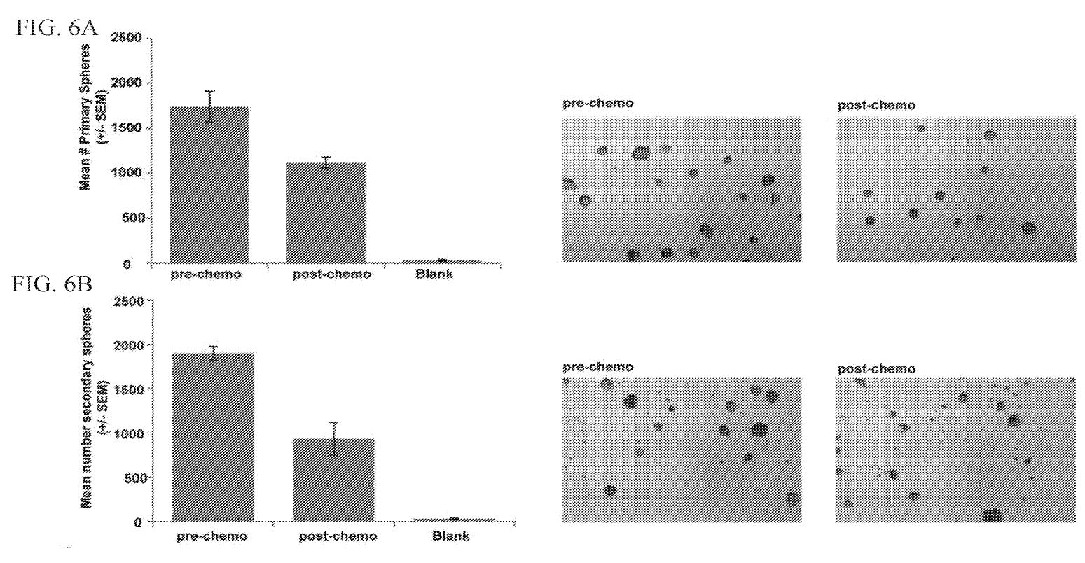

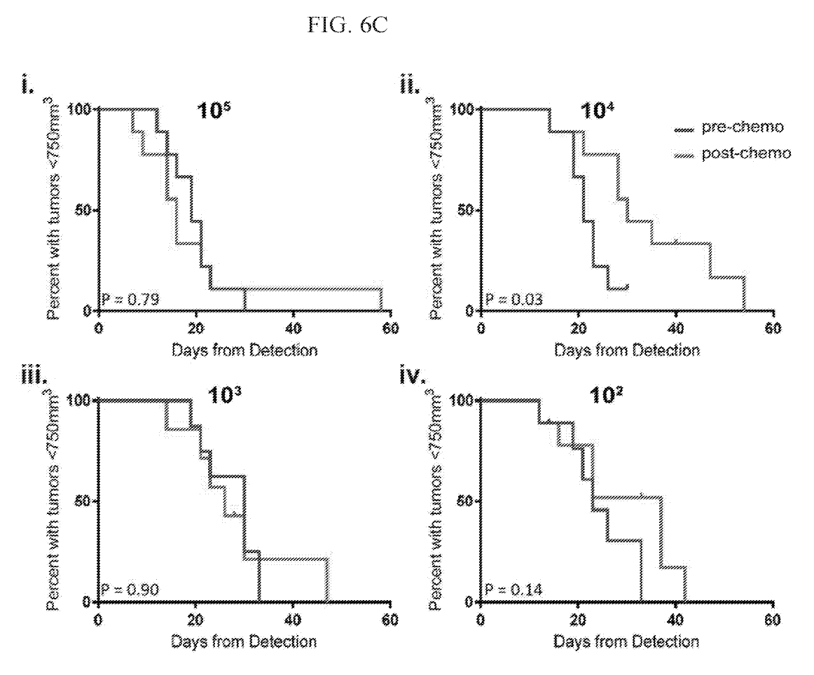

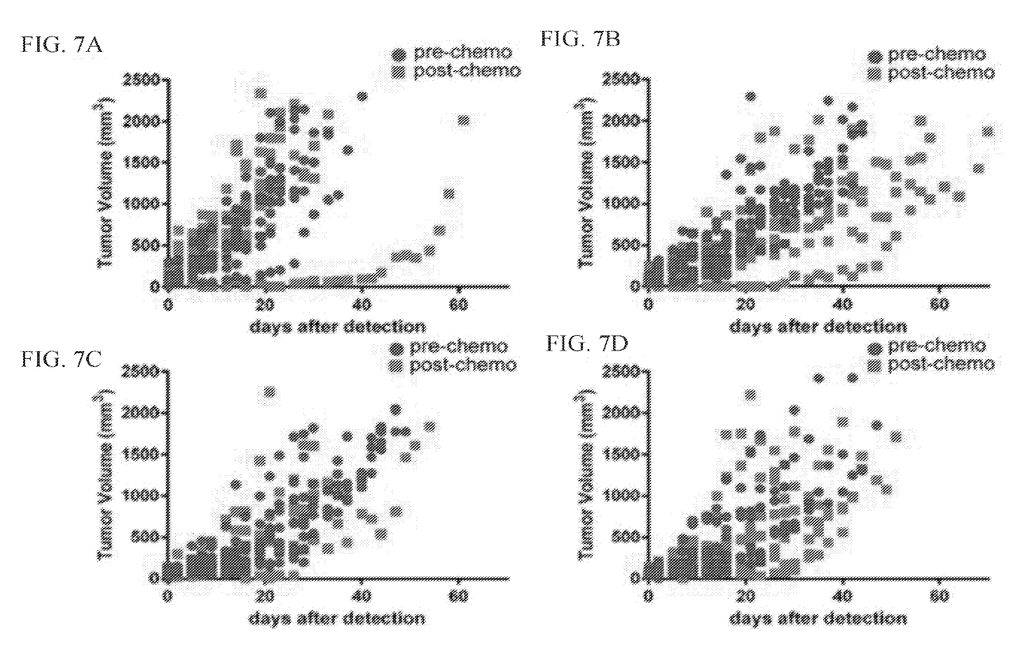

Previous studies indicate that long-term chemotherapy selection models drive the growth of cancer stem-like cells [4-8]. We therefore sought to determine if chemo-resistant TN tumor cells emanating from our short-term chemotherapy treatment model exhibit cancer stem-like properties. As shown in FIG. 6, chemo-resistant tumor cells from our model did not exhibit an increased ability to grow as non-adherent spheres, a defining property of cancer stem-like cells. In fact, they had decreased ability compared to their non-treated parental counterparts. To measure their self-renewing activity, primary spheres were dissociated into single cells, and the efficiency of secondary sphere formation was determined. As shown in Suppl. FIG. 28, chemo-resistant tumor cells from our model did not exhibit increased self-renewing activity compared to parental tumor cells. Because cancer stem-like cells exhibit increased tumor-initiating activity, we next assessed the relative tumor-initiating ability of chemo-resistant and parental triple-negative tumor cells in an orthotopic mouse model. SUM159 cells obtained pre- and post-chemotherapy were injected in a limiting dilution study into the mammary fat pad of NSG mice (10 mice/group). Tumor volumes were assessed using calipers on a weekly basis until tumors reached a size of 100 mm³, at which point they were measured every 2-3 days until volumes reached 2000 mm³. As shown in Suppl. FIG. 2C, tumor cells obtained post-chemotherapy treatment did not exhibit increased tumor-initiating activity compared to untreated TN tumor cells at any injection number. Furthermore, there were no differences in tumor growth rate between chemo-residual and parental grafts (FIG. 7).

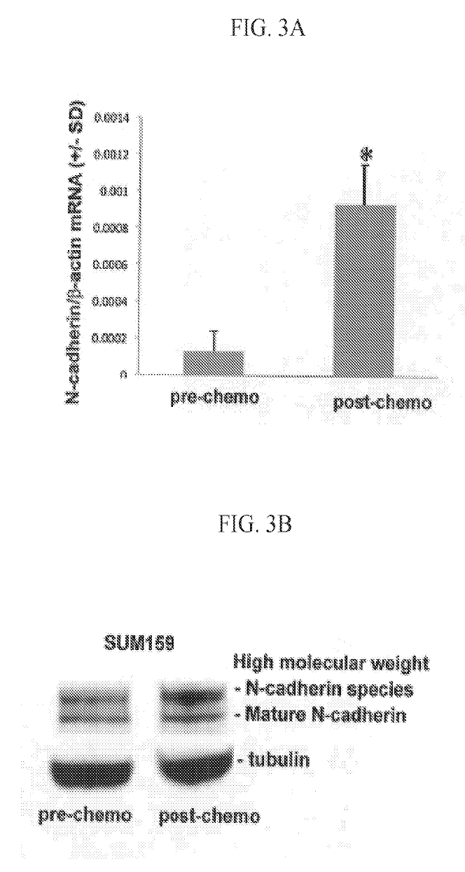

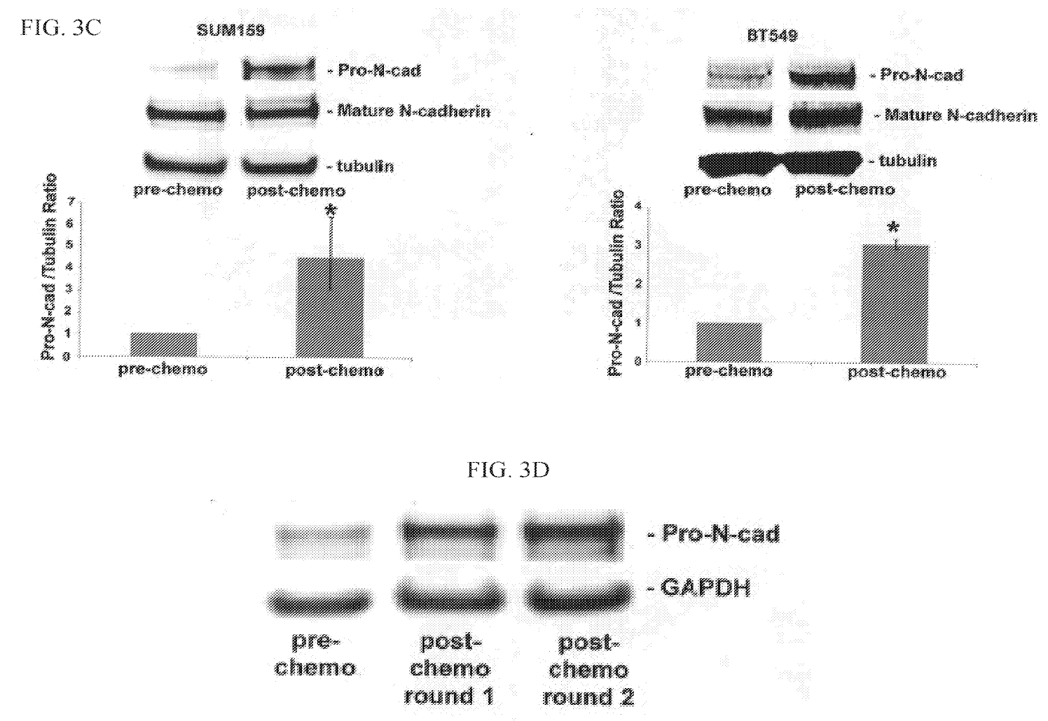

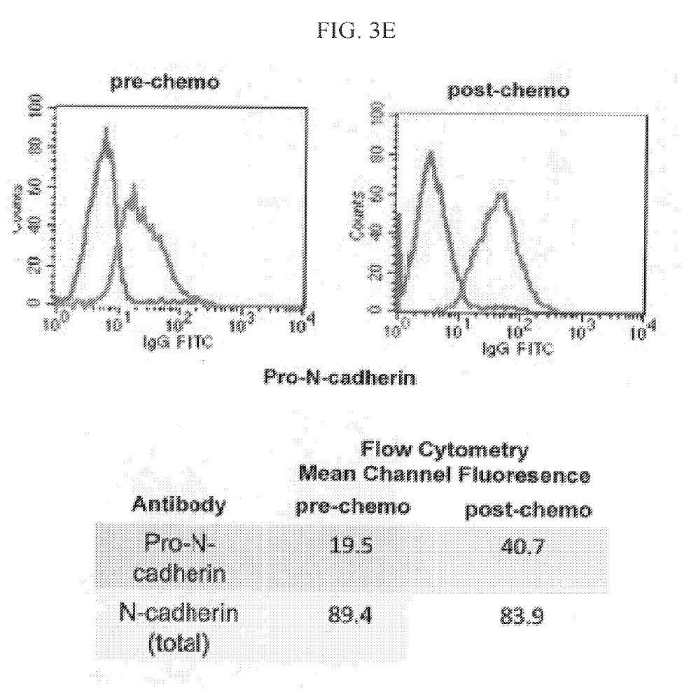

Long-term chemotherapy selection models drive an epithelial-mesenchymal transition in estrogen receptor-positive breast tumors, characterized by reduced epithelial adhesion marker (E-cadherin) and acquired mesenchymal adhesion marker (N-cadherin) expression. By contrast, triple-negative breast cancers are typically mesenchymal in nature, expressing significant N-cadherin prior to chemotherapy treatment. We performed real-time PCR to determine relative levels of N-cadherin in parental (untreated) and chemo-resistant SUM159 cells from our short term chemotherapy treatment model. As shown in FIG. 3A, SUM159 cells obtained post-chemotherapy treatment exhibited a seven-fold increase in N-cadherin mRNA levels compared to that observed in untreated SUM159 cells. Surprisingly, levels of N-cadherin protein (120 kDa) were equal in SUM159 cells obtained pre- and post-chemotherapy treatment (FIG. 3B). We did however observe that the N-cadherin antibody reacted with a higher molecular weight species, the expression of which was significantly increased in SUM159 tumor cells obtained post-chemotherapy treatment compared to parental SUM159 tumor cells (FIG. 38). Based on the knowledge that N-cadherin is synthesized as a precursor protein (pro-N-cadherin) that is cleaved by proteases to generate the mature form [11], we next investigated levels of pro-N-cadherin in TN tumor cells obtained pre- and post-chemotherapy treatment.

Chemo-resistant SUM159 and BT549 cells generated in our short term chemotherapy treatment model expressed significantly increased levels of Pro-N-cadherin compared to untreated cells, as detected using an antibody specific for this precursor N-cadherin protein (FIG. 3C). Notably, pro-N-cadherin protein levels were equal in chemo-resistant SUM159 cells exposed to either one or two rounds of short-term docetaxel treatment (FIG. 30), indicating that pro-N-cadherin expression was maintained over time in chemo-resistant cells.

In untransformed cells, only the mature form of N-cadherin, and not the precursor protein, is transported to the cell surface [11]. By contrast, recent studies indicate that pro-N-cadherin itself can be transported to the surface of tumor cells, driving an invasive phenotype [12]. Accordingly, we next investigated cell surface expression of Pro-N-cadherin in chemo-resistant and parental TN tumor cells by flow cytometry. Tumor cells obtained post-chemotherapy treatment expressed significantly increased levels of cell surface pro-N-cadherin compared to untreated tumor cells, as reflected by a 2-fold increase in the mean channel fluorescence (FIG. 3E).

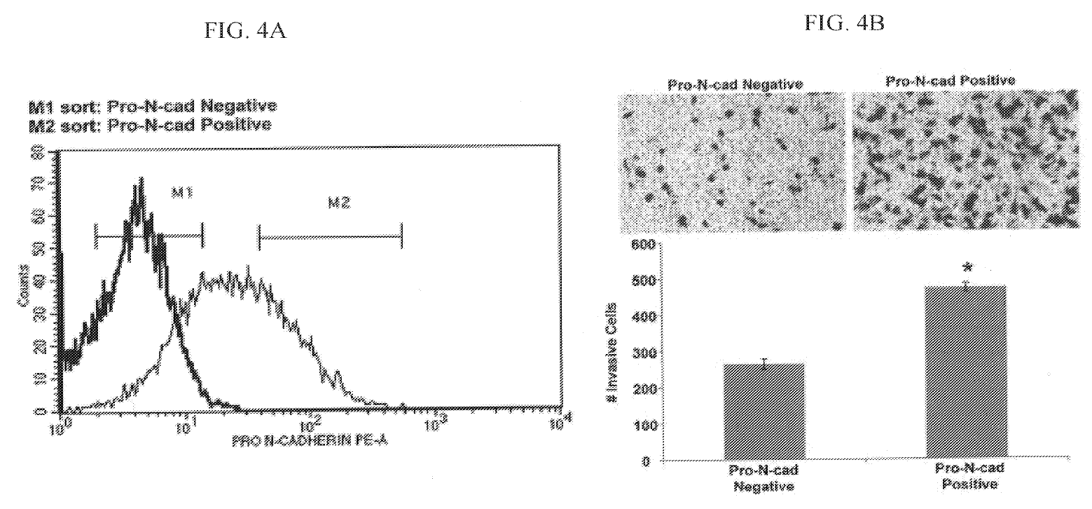

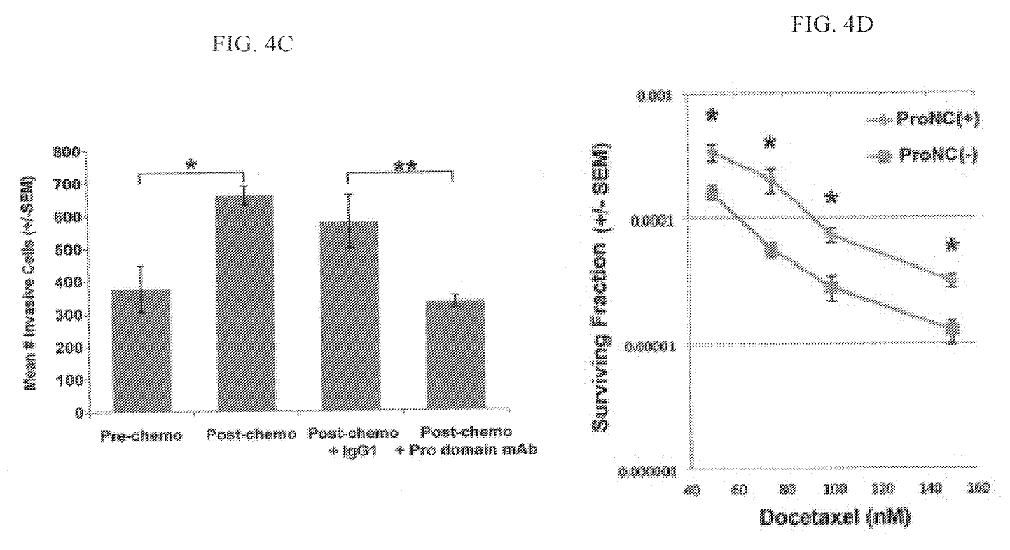

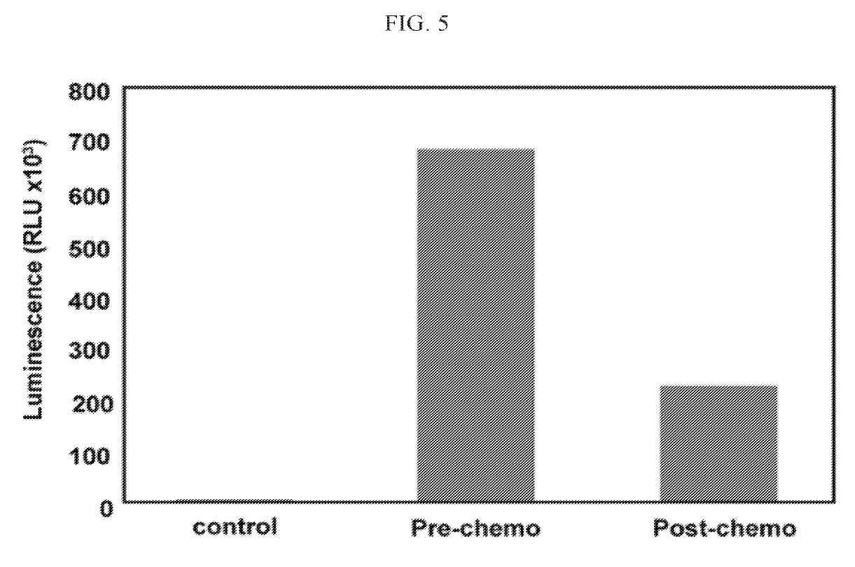

Pro-N-cadherin is expressed on the surface of melanoma and glioma cells, and contributes to their invasive behavior [12]. We hypothesized that the short-term chemotherapy treatment enriches for a resistant TN breast tumor cell subpopulation expressing cell surface pro-N-cadherin. To test this hypothesis, we performed cell sorting on untreated SUM159 TN tumor cells to separate cell-surface pro-N-cadherin-positive from cell-surface pro-N-cadherin-negative tumor cells (FIG. 4A), and investigated the relative invasive potential of these sorted populations. As shown in FIG. 4B, cell surface Pro-N-cadherin-positive SUM159 cells exhibited an approximately two-fold increase in transwell invasion compared to cell surface-Pro-N-cadherin-negative SUM159 cells. These data demonstrate that a subpopulation of SUM159 tumor cells expressing cell surface pro-N-cadherin exhibits increased invasive behavior relative to the subpopulation lacking this protein. To directly link cell surface pro-N-cadherin to the invasive behavior of chemo-resistant TN breast tumor cells, we showed that incubation of SUM159 tumor cells obtained post-chemotherapy treatment with an antibody specific for the precursor (pro) domain of N-cadherin significantly reduced their transwell invasion (FIG. 4C).

Based on the knowledge that the cell surface pro-N-cadherin-positive population was enriched by chemotherapy treatment of SUM159 cells (FIG. 4), we next investigated the relative chemo-resistance of pro-N-cadherin-positive vs pro-N-cadherin-negative sorted populations from untreated SUM159 tumor cells. As shown in FIG. 40, the pro-N-cadherin positive population was significantly more resistant to docetaxel than the pro-N-cadherin-negative sorted population. Collectively, these data indicate that: 1) a highly invasive and chemo-resistant subpopulation of TN tumor cells expresses high levels of cell surface pro-N-cadherin, and 2) this subpopulation is enriched by short-term chemotherapy treatment.

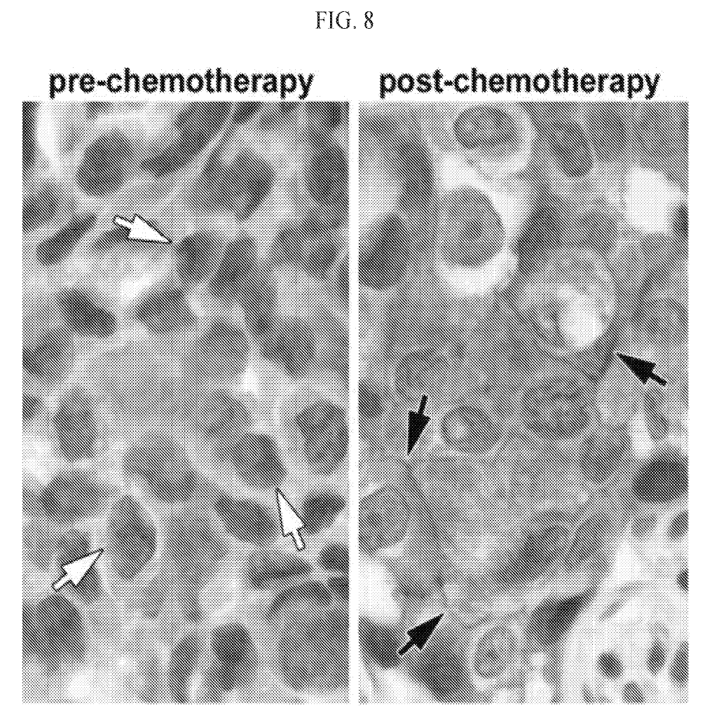

We next sought to validate our findings in chemo-residual tumor cells from TN breast cancer patients. Matched tumor biopsies were obtained from TN breast cancer patients (n=6 cases) pre- and post-neoadjuvant chemotherapy treatment. Pro-N-cadherin expression levels were assessed in these tumor tissues by immunohistochemistry. Notably, we observed nuclear/peri-nuclear pro-N-cadherin in both pre- and post-chemotherapy cases (FIG. 8; white arrows). In contrast, in the majority of post-chemotherapy cases, we detected cell membrane pro-N-cadherin staining (FIG. 8; black arrows). Specimens were scored in a blinded fashion by two pathologists for % tumor cells expressing cell membrane (surface) Pro-N-cadherin, as well as intensity of staining (Table 1). For most of the cases, cell surface Pro-N-cadherin was detected in less than 5% of the tumor cells of tissues obtained pre-chemotherapy. In five of the six cases, the percentage of tumor cells expressing cell surface pro-N-cadherin increased appreciably post-treatment (Table 1). These data: 1) support our in vitro findings, which indicate that a population of cell surface pro-N-cadherin-expressing cells is enriched by chemotherapy treatment, and 2) underscore the clinical significance of our results.

Table 1: Cell Surface Pro-N-Cadherin Expression in Triple-Negative Breast Tumors Pre- and Post-Neoadjuvant Chemotherapy Treatment.

Six triple-negative breast cancer cases exhibiting an incomplete pathologic response to neoadjuvant chemotherapy were identified from medical records under Duke Institutional Review Board approval (Protocol 47289). Pro-N-cadherin expression in formalin-fixed, paraffin embedded tissues was assessed by immunohistochemistry using Pro-N-cadherin antibody (R&D Systems). Cell surface Pro-N-cadherin scoring was performed in a blinded fashion by two pathologists. Consensus scores for % cell surface Pro-N-cadherin(+) tumor cells are shown. Five of six cases showed increased % cell surface Pro-N-cadherin(+) tumor cells post-chemotherapy (1′). One of six cases showed reduced % cell surface Pro-N-cad(+) tumor cells post-chemotherapy (w). Intensity of staining for all positive cases was +1.

TABLE 1

| | | % cell surface | % cell surface | |
|---|---|---|---|---|
| | | pro-N-cad(+) | Pro-N-cad(+) | |
| TNBC | | tumor cells | tumor cells | Trend |
| Case | Chemotherapy | pre-chemo | post-chemo | (Post vs Pre) |
| 1 | TCx4 + Ax4 | 0 | 50 | 1′ |
| 2 | ACx4 + Pacxl | 20 | 50 | 1′ |
| 3 | TACx6 | 1 | 60 | 1′ |
| 4 | ACx4 + Pacx4 | 3 | 40 | 1′ |
| 5 | ACx4 + Pacx4 | 0 | 5 | 1′ |
| 6 | ACx4 + Pacxl | 5 | 0 | w |

Discussion:

Hormone and receptor-targeted therapies are not available for TN breast cancers, which lack ER, PR and HER2 expression. Accordingly, chemotherapy is the only available treatment for women diagnosed with these aggressive tumors. Although TN tumors initially respond to chemotherapy, the response is incomplete in the majority of cases. Half of the women with an incomplete response will experience tumor recurrence within three years. Therefore, in order to develop effective therapies that reduce patient mortality, it is of the utmost importance to identify molecular determinants of TN chemo-residual disease that contribute to tumor recurrence.

Continuous, long-term chemotherapy selection models promote the growth of cancer stem-like cells [8, 13]. Likewise, chemo-residual TN tumor cells surviving 4 days paclitaxel treatment exhibit cancer stem-like cell behaviors [4, 6]. Based on these results, we were surprised to find that TN tumor cells from our short-term chemotherapy treatment model did not exhibit cancer stem-like cell behaviors (FIG. 3). We propose two possible explanations for these discrepant results. First, the chemotherapy concentrations utilized in our studies (100-fold IC₅₀) have been achieved in patients[14], but are significantly higher than the chemotherapy concentrations previously shown to promote growth of cancer stem-like cells (50% 1Csa) [4, 6]. Accordingly, we acknowledge the possibility that high but not low chemotherapy concentrations may eliminate cancer stem cells. Alternatively, cancer stem-like cells exhibit plasticity, with micro-environmental influences such as hypoxia being important for cancer stem cell maintenance [15, 16]. Thus, it is possible that maintenance of stem-like tumor cells is dependent on continuous chemotherapy exposure, contrasting with the conditions in our short-term chemotherapy treatment model.

Our studies are the first to show that chemotherapy enriches for a highly invasive tumor cell subpopulation, the maintenance of which is not dependent on continuous drug treatment. Of note, the evolution of these therapy-resistant breast tumor cells with increased invasive behavior was dependent on tumor cell exposure to high chemotherapy concentrations (100-fold IC₅₀), which eliminated 50% of the tumor cells within 2 days (data not shown). Chemotherapy removal in our model mimics the “drug holiday” that cancer patients experience after initial treatment. Our results suggest that this drug holiday may allow for the expansion of a TN tumor cell subset that is both drug resistant and highly invasive.

Our demonstration that chemotherapy selects for an aggressive tumor cell population illustrates the previously described model of oncogenic resistance [17, 18]. According to this model, drugs select for tumor cells expressing oncoproteins that drive both resistance and aggressive behaviors contributing to tumor progression. Our studies describe a novel protein in therapy-resistant triple-negative breast tumor cells (pro-N-cadherin) that is associated with these behaviors.

Results from our study expand upon previous work, which demonstrated that cancer therapeutics can promote tumor progression by influencing the microenvironment [19, 20]. In these mouse models, angiogenesis inhibitors (e.g., Sunitinib, anti-VEGFR2) were shown to establish a metastatic niche that promotes tumor metastasis [19, 20]. Notably, in these studies, the invasive tumors elicited by anti-angiogenic therapy remained invasive, even after withdrawing therapy. In another study, chemotherapy treatment of non-tumor bearing mice established a metastatic niche that enabled injected tumors to metastasize more efficiently than that which occurs in untreated mice [21]. Our studies add to these findings by showing that, in the absence of micro-environmental influences, chemotherapy drives triple-negative tumor cell invasive behavior by selecting for highly invasive tumor cell subpopulations represented infrequently in the heterogeneous tumor and characterized by the expression of cell surface pro-N-cadherin. Similar to the pro-metastatic activities of angiogenesis inhibitors reported previously, chemotherapy-driven tumor cell invasive behavior was not dependent on continuous chemotherapy treatment.

Recent molecular profiling analyses identified novel markers of triple-negative breast cancer chemo-resistance by studying genes differentially expressed in patient tumors obtained pre- and post-neoadjuvant chemotherapy treatment[22, 23]. One drawback of molecular profiling analyses is that they fail to identify determinants of resistance that are regulated at post-transcriptional or post-translational levels. In this Example, we identify a precursor form of an adhesion molecule, the expression of which is increased in chemo-residual TN tumor cells compared to untreated tumors. Cell sorting studies indicate that cells expressing high levels of this precursor protein represent a pre-existing subpopulation in the original tumor cell line that is chemo-resistant and exhibits highly invasive behavior. Because the expression level of this marker is determined by post-translational processing, DNA/RNA profiling methods would not identify this differentially expressed protein in chemo-residual tumor cells.

During the epithelial-mesenchymal transition, epithelial tumor cells undergo a cadherin-switch, losing expression of the epithelial adhesion marker E-cadherin and gaining expression of neural cadherin (N-cadherin). Acquired N-cadherin expression in these cells drives invasive and metastatic tumor cell behaviors [24-26]. N-cadherin is expressed as a precursor form (pro-N-cadherin) lacking adhesive function. A specific pro-protein convertase cleaves pro-N-cadherin in the Golgi apparatus, allowing for the adhesion molecule to be transported to the cell surface [11]. A recent study indicates that tumor cells (e.g., melanoma, brain) exhibit a unique ability to transport pro-N-cadherin, the immature form of N-cadherin, to the cell surface [12]. Notably, cell surface-expressed pro-N-cadherin drives tumor cell invasion, and pro-N-cadherin expression is directly associated with breast cancer grade [12]. This Example adds to these findings by showing that cell surface pro-N-cadherin expression is detected in triple-negative breast cancers. Furthermore, we demonstrate that triple-negative breast tumors are heterogeneous, being composed of both cell surface pro-N-cadherin-positive and -negative tumor cell subpopulations. Finally, by cell sorting, we show that TN tumor cell subsets expressing cell surface pro-N-cadherin exhibit increased invasive behavior compared to TN tumor cells lacking this precursor protein on the cell surface.

Our studies of TN breast cancer cases (n=6) obtained pre- and post-neoadjuvant chemotherapy treatment demonstrate that most patients exhibit an increased percentage of cell surface pro-N-cadherin-positive tumor cells post-treatment compared to pre-treatment (Table 1), validating our in vitro findings. All of these patients received either anthracycline or anthracycline+taxane therapy, and exhibited an incomplete pathologic response. Considering that multiple TN breast cancer subtypes have been identified [27], we hypothesize that cell surface pro-N-cadherin-positive cells may only be enriched in a subset of TN breast cancer subtypes. We further hypothesize that with follow-up, the cases with increased % cell surface pro-N-cadherin-positive cells will exhibit future tumor recurrence. This pilot data underscores the importance of performing a larger, prospective study of pro-N-cadherin expression in TN breast cancer cases pre- and post-neoadjuvant chemotherapy treatment, controlling for TN breast cancer subtype. These follow-up studies have the potential to identify a novel biomarker in a subset of TN breast cancer patients that predicts tumor recurrence. In addition, identifying cell surface pro-N-cadherin as a determinant of chemo-resistance in a subset of TN breast cancers will establish a logical therapeutic strategy for chemo-sensitizing tumors in these patients.

Pro-N-cadherin is cleaved by a specific pro-protein convertase (furin). Reduced furin levels have been reported to promote increased pro-N-cadherin expression in glioma and melanoma cells, resulting in elevated migratory/invasive behavior [12]. Of note, we did not detect reduced furin in chemo-residual TN tumor cells (data not shown), suggesting that alternative signaling drives pro-N-cadherin cell surface expression in chemo-residual TN breast tumor cells. Notably, chemo-residual TN breast tumor cells expressed significantly elevated N-cadherin mRNA levels compared to untreated TN breast tumor cells.

Not to be bound by any theory, but the elevated N-cadherin levels in chemo-resistant cells may not get processed to the mature form because of limiting amounts of the pro-protein convertase, furin.

Further, not to be bound by any theory, but we believe the mechanisms by which pro-N-cadherin expression increases invasion in TNBC may be an ability of pro-N-cadherin to prevent cell-cell adhesion, thus favoring invasion. Alternatively, considering that N-cadherin drives tumor cell invasion/metastasis by associating with FGFR1 and preventing its endocytosis [28, 29], it may be that pro-N-cadherin promotes FGFR1 signaling more efficiently than does N-cadherin, resulting in increased tumor cell invasion.

In summary, our work indicates that TN tumor cells exposed to short-term chemotherapy exhibit increased invasive behavior relative to the untreated tumor cells. We also identify a precursor form of an adhesion protein, the expression of which is upregulated on chemo-resistant TN tumor cells. Considering that the establishment of distant tumor recurrence is highly dependent on chemo-residual tumor cell invasion, we suggest that this precursor adhesion protein may be a central determinant of TN breast cancer recurrence, a topic of current investigation.

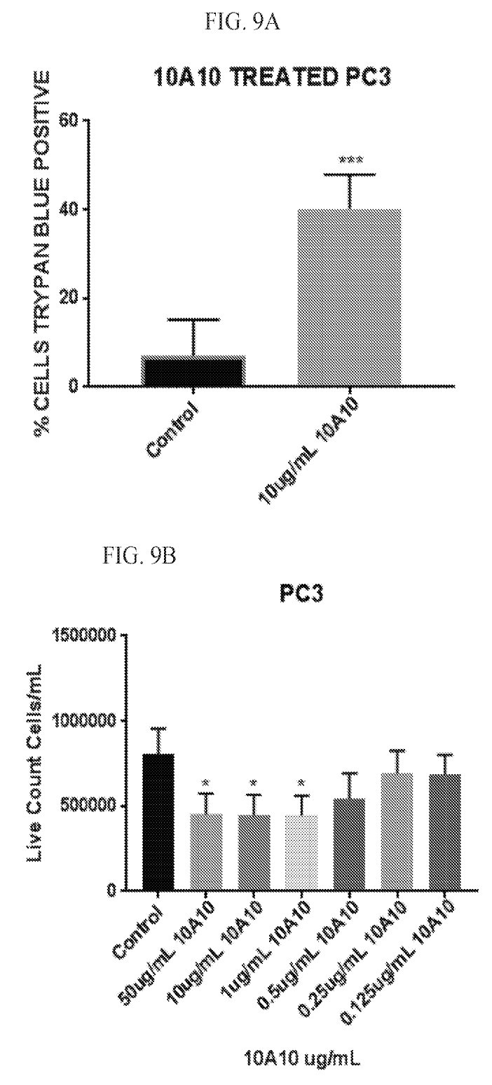

Further, FIGS. 9A and 9B demonstrate that the antibody specific for pro-N-cadherin is able to kill PC3 cells, which are a metastatic prostate cancer cell line.

Conclusions:

TN tumor cells surviving short-term chemotherapy treatment exhibit increased behavior compared to untreated tumor cells due to their increased expression of cell surface precursor N-cadherin. Cell surface precursor N-cadherin is expressed on chemo-residual tumor cells from TNBC patients. This demonstrates that cell surface pro-N-cadherin in TN tumor cells may predict future tumor recurrence, and that this cell surface protein may be targeted to eliminate chemo-residual TNBC disease/prevent recurrence.

Materials and Methods:

Cell Culture:

SUM159 and BT549 triple-negative breast tumor cells were obtained from Duke Cell Culture Facility in 2010. Both cell lines were authenticated (August 2015) with STR profiling at the Duke DNA facility using GenePrint 10 kit (Promega). SUM159 cells were maintained in Ham's F-12 medium containing 5% heat-inactivated FBS, 5 μg/L insulin, and 1μg/ml hydrocortisone. BT549 cells were maintained in RPMI 1640 containing 10% heat-inactivated FBS, 1 μg/ml insulin, 10 mM HEPES, 1 mM pyruvate, and 2.5 g/L glucose.

Short-Term Chemotherapy Treatment Model:

SUM159 or BT549 triple-negative tumor cells were cultured for 2 days in Docetaxel (100 nM). After Docetaxel removal, chemo-residual tumor cells were allowed to recover in drug-free complete medium for an additional 16 d. At this time, colonies emanating from chemo-residual tumor cells were harvested with EDTA and expanded as a monolayer for one passage prior to analysis of chemo-residual tumor cell signaling/invasive behavior. To generate chemo-residual tumor cells exposed to two rounds of docetaxel, cells emanating from round 1 of treatment (described above) were subjected to 2 day docetaxel (100 nM) treatment as above. After docetaxel removal, chemo-residual tumor cells were allowed to recover in drug-free complete medium for an additional 16 d. Colonies were harvested with EDTA and expanded as a monolayer, as above.

Thymidine Incorporation Assay:

Cells were seeded into a 96 well tissue culture plate at a seeding density of 5000 cell/well) (×6). After 12 hours incubation at 37° C. (5% C0₂), [Methyl-3H]-Thymidine (Perkin Elmer; 0.5 μCi/well) was added. After incubation at 37° C. (5% C0₂) for 4 h, medium was removed, and cells were harvested onto glass fiber filters (FilterMat, Skatron Instruments). [3H]-Thymidine incorporation was measured as counts per minute (CPM) using a Tricarb 2100TR Liquid Scintillation Analyzer (Packard). Mean thymidine incorporation from 6 wells (+/−SEM) was calculated for each cell population.

Matrigel Transwell Invasion Assay:

Wells (Costar 3422, 24 well, Sum plate) were coated at 4° C. overnight with BME Pathclear Matrigel (Trevigen 3442-005-01; 5 μg/well). Cells were harvested with 5 mM EDTA/HBSS (Gibco), and washed 3× with 10 ml culture medium+ 0.1% BSA. After counting, cells were seeded at 50000 or 25000 cells in 100 ul media+pen/strep+0.1% BSA into the top chamber of Matrigel transwells (triplicate wells for each cell line). For some experiments (FIG. 5C), pro-N-cadherin monoclonal antibody [30] (kindly provided by Dr. James Wahl) or isotype control antibody (mouse lgG1, Sigma) were added with cells to top chambers. 600 μl complete media with serum was added to the bottom chamber of each transwell as a source of chemo-attractant. Plates were incubated at 37° C. at 5% CO₂ After 4 h plates were removed and the tops of the transwell inserts were wiped with a Q-tip to remove cells. The inserts were fixed with cold (−20 C) Methanol for 10 minutes and then stained with 0.2 mg/ml Crystal Violet in 2% Ethanol for 10 min. Inserts were left to air dry overnight and photographed at 100×. The number of invaded cells from 5 fields per insert were counted using cell count in Image J software (NIH). Mean# invasive cells from three triplicate wells (+/−SD) was determined for each cell population.

Tail Vein Injection Model:

Immune compromised Nod Seid Gamma (NSG) mice (n=20) were obtained from an in-house breeding colony (Duke Cancer Center) and divided into two groups (n=10 per group). The first group of mice was injected via tail vein with 1 as parental tumor cells/mouse. The second group was injected with 1 as chemo-residual cells/mouse. Subsequent growth of metastatic colonies was monitored over time by in vivo bioluminescent imaging. 34 days post-graft, mice were euthanized, and lungs were fixed and removed for quantification of macroscopic metastatic burden. Median number of metastatic nodules was determined for each group.

Tumor Initiating and Growth Model:

NSG mice were divided into eight groups (n=10 mice/group). Cells (pre-chemo or post-chemo) were grafted orthotopically into the inguinal mammary fat pad of NSG mice at dilutions of 10⁵, 104, 10³, or 10² cells per mouse. Resulting tumors were tracked by direct caliper measurements.

Mammosphere Culture:

Cells were seeded into Mammocult media (Stem Cell Tech., #05620) supplemented with 1% Methylcellulose (Sigma #M0430), pen/strep (Gibco), Heparin (Stem Cell Tech., #07980; 4 μg/ml), and Hydrocortisone (1.0 μg/ml). Sphere assays were setup in Costar 6 Well Ultra Low Attachment (#3471) plates in triplicate. Cells were seeded (20000 cells/well) into each well in complete Mammocult media and incubated at 37° C. in 5% C02. Number of spheres (2:50 μm) was counted after 7 d using Gel Count. Data were reported as number of spheres from 3 wells (+/−SEM). For secondary spheres, primary spheres were trypsinized, washed with regular media, and seeded at 20000 cells/well as above. Sphere counts were determined on d7, as above.

Cytosolic and Nuclear Protein Extraction:

Cells were harvested using 2 mM EDTA/HBSS and washed 2×14 with HBSS (Gibco). Cytosolic extracts were prepared using lysis buffer [10 mM Hepes, pH 7.6, 10 mM KCl, 1.5 mM MgCb, 0.5% NP40, Halt Phosphatase/Protease Inhibitor (Pierce), PMSF (1 mM)]. Cells were lysed on ice for 20 minutes then centrifuged at 3500 rpm for 5 minutes at 4° C. Supernatant containing cytosolic proteins was removed and stored at −80 C. Nuclear proteins were extracted from the pellet on ice for 15 min using nuclear extraction buffer [1% SOS in 50 mM Tris pH 7.5, Halt Phosphatase/Protease Inhibitor, 1 mM PMSF, 0.5 ul Benzonase (Sigma)]. Extracts were centrifuged at 4° C. at 14000 rpm for 10 min. Supernatant containing nuclear proteins was removed and stored at −80° C. Protein concentrations were determined using BCA Protein Assay Kit (Pierce).

Western Blotting:

1× Gel Loading Buffer with beta mercaptoethanol (5 mM) was added to 40 ug of lysate. Samples were boiled for 5 min, and then loaded into a 10% NuPage Bis-Tris Gel (Invitrogen). Proteins were transferred to nitrocellulose in cold 1× Tris/Glycine/10% Methanol with an ice pack at 0.3 amps for 50 min. Membranes were then blocked with Rockland Blocking Buffer (Rockland) for 1 hour. Membranes were probed with primary antibodies overnight at 4 C with rocking. Membranes were then washed once with PBS/0.5% Tween 20. Fresh Blocking Buffer was added and IR-dye-conjugated secondary antibodies were added at room temperature with rocking for 40 to 50 minutes. Membranes were washed 3× with PBS/0.5% Tween 20 and 3× with PBS. Proteins were visualized with an Odyssey Infrared Imaging System. Bands were quantified using Image J software (NIH).

Pro-N-Cadherin Sorting:

SUM159 cells were harvested with 2 mM EDTA in HBSS. Harvested cells were washed in wash buffer (HBSS/0.5% BSA, Pen/Strep), and then incubated with PE-conjugated Pro-N-cadherin (20 ul/2×10e6 cells) for 45 minutes at 4 C. Fifteen million cells were typically stained with Pro-N-cadherin-PE antibody. Cells were washed in wash buffer, resuspended in complete media, and placed through a 30 μm cell strainer to obtain single cells. 7-AAD (5 μL per million cells) was added immediately before cell sorting. Live cells (7-AAD negative) were sorted by pro-N-cadherin intensity (top 30%=high and bottom 30%=low/negative).

Pro-N-Cadherin IHC:

TN breast cancer patients treated with neoadjuvant chemotherapy that exhibited an incomplete pathologic response were identified from medical records under Duke Institutional Review Board approval (Protocol 47289). Retrospectively collected tumor biopsies (obtained pre-chemotherapy) and biopsies/resections (obtained post-chemotherapy) from these patients were retrieved. Formalin-fixed, paraffin-embedded tissues were subjected to pro-N-cadherin immunohistochemistry. Slides were baked at 60° C. for 1 hour, and deparaffinized in xylene followed by 100% ethanol. Antigen retrieval was performed in subboiling Citrate buffer at 100° C. for 40 min. Pro-N-cadherin staining was performed in an autostainer according to the following program: Endogenous Peroxidase Quench (R&D-Peroxidase), 5 min; Protein block (R&D Serum block, Avidin block, Biotin block), 15 min each blocking step; Pro-N-cadherin antibody (R&D Systems Sheep anti-pro-N-cadherin (0.4 μg/ml), 2 hour; secondary detection kit (R&D HRP sheep detection kit with DAB), 45 min; DAB, 7 min; hematoxylin, 1 min; Bluing, 1 min. Slides were then placed in water and dehydrated in ethanol to xylene before adding a coverslip. Formalin-fixed, paraffin-embedded slides of SUM159 breast tumor cells [Pro-N-cadherin (+)] and MCF7 breast tumor cells (Pro-N-cadherin (−)] were prepared as positive and negative controls respectively for Pro-N-cadherin reactivity.

IHC Scoring:

Two pathologists (EP, GD) (blinded to patient samples) assigned scores for percent tumor cells positive for cell surface (membrane) pro-N-cadherin staining, as well as intensity of staining (+1, +2).

Clonogenics Assay:

Pro-N-cadherin-positive and Pro-N-cadherin-negative populations were sorted from untreated SUM159 cells, seeded into 6 well plates (4 wells per condition) at varying cell densities, and incubated at 37° C., 5% C02 overnight. The next day, culture medium was removed and treatments were added [no treatment, Docetaxel (50 nM, 75 nM, 100 nM and 150 nM]. After 48 h, treatments were removed, cells were washed with HBSS, and fresh culture medium (lacking drug) was added. Every three days, culture medium was changed and plates were examined for colony formation. To stain colonies, plates were fixed in 10% Acetone/10% Methanol solution for 10 min. Colonies were stained with 2% Crystal Violet for 30 minutes. The plates are then washed with water and allowed to air dry. Plates were imaged on GelCount and colonies were counted with Image J.

### LIST OF ABBREVIATIONS

TN, triple-negative

TNBC, triple-negative breast cancer

Pro-N-cadherin, precursor neural-cadherin

FGFR1, fibroblast growth factor receptor 1

### REFERENCES

1. Liedtke C, Mazouni C, Hess K R, Andre F, Tordai A, Mejia J A, Symmans W F, Gonzalez-Angulo A M, Hennessy B, Green M, Cristofanilli M, Hortobagyi G N and Pusztai L. Response to neoadjuvant therapy and long-term survival in patients with triple-negative breast cancer. J Clin Oneal. 2008; 26(8): 1275-1281.
2. Moore N, Houghton J and Lyle S. Slow-cycling therapy-resistant cancer cells. Stem Cells Dev. 2012; 21(10):1822-1830.
3. Sharma S V, Lee D Y, Li B, Quinlan M P, Takahashi F, Maheswaran S, McDermott U, Azizian N, Zou L, Fischbach M A, Wong K K, Brandstetter K, Wittner B, Ramaswamy S, Classon M and Settleman J. A chromatin-mediated reversible drug-tolerant state in cancer cell subpopulations. Cell. 2010; 141(1):69-80.
4. Bhola N E, Balko J M, Dugger T C, Kuba M G, Sanchez V, Sanders M, Stanford J, Cook R S and Arteaga C L. TGF-beta inhibition enhances chemotherapy action against triple-negative breast cancer. J Clin Invest. 2013; 123(3):1348-1358.
5. Li X, Lewis M T, Huang H, Gutierrez C, Osborne C K, Wu M F, Hilsenbeck S G, Pavlick A, Zhang X, Chamness G C, Wong H, Rosen J and Chang J C. Intrinsic resistance of tumorigenic breast cancer cells to chemotherapy. J Natl Cancer Inst. 2008; 100(9):672-679.
6. Samanta D, Gilkes O M, Chaturvedi P, Xiang Land Semenza G L. Hypoxia-inducible factors are required for chemotherapy resistance of breast cancer stem cells. Proceedings of the National Academy of Sciences of the United States of America. 2014 111(50):E5429-5438
7. Zhang S, Mercado-Uribe I and Liu J. rumor stroma and differentiated cancer cells can be originated directly from polyploid giant cancer cells induced by paclitaxel. Intl Cancer. 2014; 134(3):508-518.
8. Achuthan S, Santhoshkumar T R, Prabhakar J, Nair S A and Pillai M R. Drug-induced senescence generates chemoresistant stemlike cells with low reactive oxygen species. J Biol Chem. 2011; 286(43):37813-37829.
9. Li S, Kennedy M, Payne S, Kennedy K, Seewaldt V L, Pizzo S V and Bachelder R E. Model of tumor dormancy/recurrence after short-term chemotherapy PLoS One. 2014; 9(5):e98021
10. Kuczynski E A, Sargent D J, Grothey A and Kerbel R S. Drug rechallenge and treatment beyond progression—implications for drug resistance. Nat Rev Clin Oneal. 2013; 10(10):571-587.
11. Reines A, Bernier L P, McAdam R, Belkaid W, Shan W, Koch A W, Seguela P, Colman D R and Dhaunchak A S. N-cadherin prodomain processing regulates synaptogenesis. The Journal of neuroscience: the official journal of the Society for Neuroscience. 2012; 32(18):6323-6334.
12. Maret D, Gruzglin E, Sadr M S, Siu V, Shan W, Koch A W, Seidah N G, Del Maestro R F and Colman D R. Surface expression of precursor N-cadherin promotes tumor cell invasion. Neoplasia. 2010; 12(12):1066-1080.
13. Calcagno A M, Salcido C D, Gillet J P, Wu C P, Fostel J M, Mumau M D, Gottesman M M, Varticovski L and Ambudkar S V. Prolonged drug selection of breast cancer cells and enrichment of cancer stem cell characteristics. J Natl Cancer Inst. 2010; 102(21):1637-1652.
14. Brunsvig P F, Andersen A, Aamdal S, Kristensen V and Olsen H. Pharmacokinetic analysis of two different docetaxel dose levels in patients with non-small cell lung cancer treated with docetaxel as monotherapy or with concurrent radiotherapy. BMC Cancer. 2007; 7:197.
15. Chaffer C L, Marjanovic N D, Lee T, Bell G, Kleer C G, Reinhardt F, D'Alessio A C, Young R A and Weinberg R A. Poised chromatin at the ZEB1 promoter enables breast cancer cell plasticity and enhances tumorigencity. Cell. 2013; 154(1):61-74.
16. Heddleston J M, Li Z, Mclendon R E, Hjelmeland A B and Rich J N. The hypoxic microenvironment maintains glioblastoma stem cells and promotes reprogramming towards a cancer stem cell phenotype. Cell cycle. 2009; 8(20):3274-3284.
17. Blagosklonny M V. Oncogenic resistance to growth-limiting conditions. Nat Rev Cancer. 2002; 2(3):221-225.
18. Blagosklonny M V. Why therapeutic response may not prolong the life of a cancer patient: selection for oncogenic resistance. Cell cycle. 2005; 4(12):1693-1698.
19. Paez-Ribes M, Allen E, Hudock J, Takeda T, Okuyama H, Vinals F, Inoue M, Bergers G, Hanahan D and Casanovas 0. Antiangiogenic therapy elicits malignant progression of tumors to increased local invasion and distant metastasis. Cancer Cell. 2009; 15(3):220-231.
20. Ebos J M, Lee C R, Cruz-Munoz W, Bjarnason G A, Christensen J G and Kerbel R S. Accelerated metastasis after short-term treatment with a potent inhibitor of tumor angiogenesis. Cancer Cell. 2009; 15(3):232-239.
21. Daenen L G, Roodhart J M, van Amersfoort M, Dehnad M, Roessingh W, Ulfman L H, Derksen P W and Voest E E. Chemotherapy enhances metastasis formation via VEGFR-1-expressing endothelial cells. Cancer Res. 2011; 71(22):6976-6985.
22. Balko J M, Cook R S, Vaught D B, Kuba M G, Miller T W, Bhola N E, Sanders M E, Granja-lngram NM, Smith iJ, Meszoely I M, Salter J, Dowsett M, Stemke-Hale K, Gonzalez-Angulo A M, Mills G B, Pinto J A, et al. Profiling of residual breast cancers after neoadjuvant chemotherapy identifies DUSP4 deficiency as a mechanism of drug resistance. Nature medicine. 2012; 18(7):1052-1059.
23. Balko J M, Giltnane J M, Wang K, Schwarz L J, Young C D, Cook R S, Owens P, Sanders M E, Kuba M G, Sanchez V, Kurupi R, Moore P D, Pinto J A, Doimi F D, Gomez H, Horiuchi D, et al. Molecular profiling of the residual disease of triple-negative breast cancers after neoadjuvant chemotherapy identifies actionable therapeutic targets. Cancer discovery. 2014; 4(2):232-245.
24. Nieman M T, Prudoff R S, Johnson K R and Wheelock M J. N-cadherin promotes motility in human breast cancer cells regardless of their E-cadherin expression. The Journal of cell biology. 1999; 147(3):631-644.
25. Hazan R B, Phillips G R, Qiao R F, Norton Land Aaronson S A. Exogenous expression of N-cadherin in breast cancer cells induces cell migration, invasion, and metastasis. The Journal of cell biology. 2000; 148(4):779-790.
26. Hulit J, Suyama K, Chung S, Keren R, Agiostratidou G, Shan W, Dong X, Williams T_{M}, Lisanti M P, Knudsen K and Hazan R B. N-cadherin signaling potentiates mammary tumor metastasis via enhanced extracellular signal-regulated kinase activation. Cancer Res. 2007; 67(7):3106-3116.
27. Lehmann B O, Bauer J A, Chen X, Sanders M E, Chakravarthy A B, Shyr Y and Pietenpol J A. Identification of human triple-negative breast cancer subtypes and preclinical models for selection of targeted therapies. The Journal of clinical investigation. 2011; 121(7):2750-2767.
28. Qian X, Anzovino A, Kim S, Suyama K, Yao J, Hulit J, Agiostratidou G, Chandiramani N, McDaid H M, Nagi C, Cohen H W, Phillips G R, Norton L and Hazan R B. N-cadherin/FGFR promotes metastasis through epithelial-to-mesenchymal transition and stem/progenitor cell-like properties. Oncogene. 2014; 33(26):3411-3421.
29. Suyama K, Shapiro I, Guttman Mand Hazan R B. A signaling pathway leading to metastasis is controlled by N-cadherin and the FGF receptor. Cancer Cell. 2002; 2 (4):301-314.
30. Wahl J K, 3rd, Kim Y J, Cullen J M, Johnson K R and Wheelock M J. N-cadherin-catenin complexes form prior to cleavage of the proregion and transport to the plasma membrane. J Biol Chem. 2003; 278(19):17269-17276.

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present disclosure described herein are presently representative of preferred embodiments, are exemplary, and are not intended as limitations on the scope of the invention. Changes therein and other uses will occur to those skilled in the art which are encompassed within the spirit of the invention as defined by the scope of the claims.

### SEQUENCE LISTING STATEMENT

The application includes the sequence listing that is concurrently filed in computer readable form. This sequence listing is incorporated by reference herein.

What is claimed is:

1. A method of determining the risk of, prognosis of, and/or diagnosis of chemo-residual tumor growth in a subject following initial treatment comprising quantifying the amount of at least one biomarker present in a biological sample derived from the subject, wherein the biomarker is associated with chemo-residual tumor growth.

2. A method of predicting chemo-residual tumor cell growth in a subject with cancer having received treatment comprising (a) obtaining a biological sample from a subject; (b) determining the expression level of one or more biomarkers that are associated with chemo-residual tumor cell growth in the biological sample; (c) comparing the expression level of the biomarker(s) in the biological sample with that of a control, wherein the presence of one or more of the biomarkers in the sample that is in an amount greater than that of the control indicates the risk of chemo-residual tumor cell growth.

3. The method of claim 2, wherein the method further comprises (d) administering appropriate anti-cancer therapy if one or more of the biomarkers are expressed.

4. A method of detecting chemo-residual tumor growth in a subject comprising detecting the amount of at least one biomarker present in a biological sample derived from the subject, wherein the biomarker is associated with chemo-residual tumor growth.

5. The method of claim 4, wherein the detecting of at least one biomarker associated with chemo-residual tumor growth comprises comparing the expression level of the biomarker in the biological sample with that of a control, wherein the presence of one or more of the biomarkers in the sample that is in an amount greater than that of the control indicates the presence of chemo-residual tumor cells.

6. The method of claim 4 or 5, wherein the method comprises prior to detecting a step of treating the subject with at least one round of chemotherapy prior to detecting the amount of the at least one biomarker in the sample.

7. The method as in any one of the preceding claims, wherein the biomarker comprises pro-N-cadherin.

8. The method as in any one of the preceding claims in which the tumor comprises breast cancer.

9. The method according to claim 8 in which the tumor comprises triple negative breast cancer (TNBC).

10. The method of any one of the preceding claims, wherein the tumor is a metastatic tumor.

11. The method as in any of the preceding claims in which the subject is a mammal.

12. The method according to claim 11, in which the subject is a human.

13. The method as in any one of the preceding claims, wherein the biological sample is selected from the group consisting of tissues, cells, biopsies, blood, lymph, serum, plasma, urine, saliva, mucus, and tears.

14. The method according to claim 13, wherein the sample comprises biopsies.

15. The method of any one of claims 1-3, wherein the initial treatment is one or more treatments of chemotherapy.

16. The method of any one of claims 3, 7-15, wherein the appropriate anti-cancer therapy is an antibody to pro-N-cadherin.

17. The method of claim 16, wherein the method comprises administering the antibody in combination with one or more anti-cancer therapies.

18. A method of treating a subject having chemo-residual tumor cell growth comprising administering to the subject a therapeutically effective amount of an antibody specific for a biomarker associated with chemo-residual tumor cell growth.

19. The method according to claim 18, wherein the antibody comprises an antibody capable of binding to pro-N-cadherin.

20. The method according to claim 18 or 19, wherein the antibody comprises a monoclonal antibody (mAb).

21. The method of any one of claims 18-20, wherein the method comprises before the administration step a determining step, the determining step comprising: (a) obtaining a sample from the subject; and (b) detecting the presence of a biomarker within the sample, wherein the presence of the biomarker indicates the presence of chemo-residual tumor cells.

22. The method of claim 21, wherein the biomarker is pro-N-cadherin.

23. The method of any one of claims 18-22, wherein the antibody may be administered in combination with a therapeutically effective amount one or more anti-cancer therapies.

24. A method of treating, reducing or inhibiting metastatic cancer growth in a subject, the method comprising
administering to the subject a therapeutically effective amount an antibody specific to a biomarker for chemo-resistant tumor cells, wherein the administration reduces or inhibits metastatic cancer growth in the subject.

25. The method of claim 24, wherein the antibody is administered with a therapeutically effective amount of an anti-cancer drugs.

26. The method of claim 24, wherein the antibody is covalently or non-covalently linked to an anti-cancer therapy.

27. The method of any one of claims 24-26, wherein the method comprises detecting the biomarker specific for chemo-resistant tumor cells in a sample from the subject.

28. A method of targeting a cancer therapy to chemo-resistant tumor cells within a subject, the method comprising:
(a) detecting a biomarker specific for chemo-resistant tumor cells in a sample from the subject; and
(b) administering an effective amount of an antibody specific to the biomarker, wherein the antibody targets the chemo-resistant tumor cells within the subject.

29. The method of claim 28, wherein the biomarker is pro-N-cadherin.

30. The method of claim 28 or 29, wherein the antibody is administered with an anti-cancer therapy.

31. The method of claim 30, wherein the anticancer drug and the antibody are covalently or noncovalently linked.


SEQUENCE LISTING (1 sequence)

SEQ ID NO: 1 - protein, 159 aa, Homo sapiens
MCRIAGALRTLLPLLAALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKR